SEQUENCE LISTING (14 sequences)

SEQ ID NO: 1 - DNA, 1958 bp, Homo sapiens
aaaagtagctggagttaggtcatttgattttatactctgtactcaagactgctcctctctgccgactacaacagattggagccatggctttggagcagaaccagtcaacagattattattatgaggaaaatgaaatgaatggcacttatgactacagtcaatatgaactgatctgtatcaaagaagatgtcagagaatttgcaaaagttttcctccctgtattcctcacaatagttttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgtatcagcatagacagatatgtggcagtaactaaagtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacagcaaggacactcatgaagatgccaaacattaaaatatctcgacccctaaaagttctgctcacagtcgttatagttttcattgtcactcaactgccttataacattgtcaagttctgccgagccatagacatcatctactccctgatcaccagctgcaacatgagcaaacgcatggacatcgccatccaagtcacagaaagcatcgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaaaggtaaaactgctctgccttttgcttggatacatatgaatgatgctttcccctcaaataaaacatctgcattattctgaaactcaaatctcagacgccgtggttgcaacttataataaagaatgggttgggggaagggggagaaataaaagccaagaagaggaaacaagataataaatgtacaaaacatgaaaattaaaatgaacaatataggaaaataattgtaacaggcataagtgaataacactctgctgtaacgaagaagagctttgtggtgataattttgtatcttggttgcagtggtgcttatacaaatctacacaagtgataaaatgacacagaactatatacacacattgtaccaatttcaatttcctggttttgacattatagtataattatgtaagatggaaccattggggaaaactgggtgaagggtacccaggaccactctgtaccatctttgtaacttcctgtgaatttataataatttcaaaataaaacaagttaaaaaaaaacccactatgctataagttaggccatctaaaacagattattaaagaggttcatgttaaaaggcatttataattatttttaattatctaagttttaatacaagaacgatttccctgcataattttagtacttgaataagtatgcagcagaactccaactatcttttttcctgttttttttaaatttgtaagtaattttataaaatccacctcctccaaaaaagcaataaaaaaaaaacaaactataaaaaaaaaaaaaaaaaaaaaaaaaaaaaaaaaaaaaaaaaaaaaaaaaaaaaaaaaaaaaa

SEQ ID NO: 2 - protein, 350 aa, Homo sapiens
MALEQNQSTDYYYEENEMNGTYDYSQYELICIKEDVREFAKVFLPVFLTIVFVIGLAGNSMVVAIYAYYKKQRTKTDVYILNLAVADLLLLFTLPFWAVNAVHGWVLGKIMCKITSALYTLNFVSGMQFLACISIDRYVAVTKVPSQSGVGKPCWIICFCVWMAAILLSIPQLVFYTVNDNARCIPIFPRYLGTSMKALIQMLEICIGFVVPFLIMGVCYFITARTLMKMPNIKISRPLKVLLTVVIVFIVTQLPYNIVKFCRAIDIIYSLITSCNMSKRMDIAIQVTESIALFHSCLNPILYVFMGASFKNYVMKVAKKYGSWRRQRQSVEEFPFDSEGPTEPTSTFSI

SEQ ID NO: 3 - DNA, 23 bp, Homo sapiens
actaccaacaggttggtacttta

SEQ ID NO: 4 - DNA, 22 bp, Homo sapiens
ctttgccatctagagtggagcc

SEQ ID NO: 5 - DNA, 82 bp, Artificial Sequence
  misc_feature (1)...(82): n = A,T,C or G
ctttctattctcactccgctgaannsnnsnnsnnsnnsnnsnnsnnsnnsnnsnnsnnsnnsnnsccgcctccacctccacc

SEQ ID NO: 6 - DNA, 93 bp, Artificial Sequence
  misc_feature (1)...(93): n = inosine
ggccggtggaggtggaggcggnnnnnnnnnnnnnnnnnnnnnnnnnnnnnnnnnnnnnnnnnnnnnttcagcggagtgagaatagaaaggtac

SEQ ID NO: 7 - DNA, 36 bp, Artificial Sequence
  primer
gctgcccgagagatctgtatatatgagtaaacttgg

SEQ ID NO: 8 - DNA, 36 bp, Artificial Sequence
  primer
gcaggctcgggaattcgggaaatgtgcgcggaaccc

SEQ ID NO: 9 - DNA, 21 bp, Artificial Sequence
  mutagenic oligonucleotides
aaacttcctcatgaaaaagtc

SEQ ID NO: 10 - DNA, 25 bp, Artificial Sequence
  mutagenic oligonucleotides
agaatagaaaggtaccactaaagga

SEQ ID NO: 11 - DNA, 39 bp, Artificial Sequence
  mutagenic oligonucleotides
tttagtggtacctttctattctcactcggccgaaactgt

SEQ ID NO: 12 - DNA, 24 bp, Artificial Sequence
  mutagenic oligonucleotides
aaagcgcagtctctgaatttaccg

SEQ ID NO: 13 - DNA, 22 bp, Artificial Sequence
  primers
tcgaaagcaagctgataaaccg

SEQ ID NO: 14 - DNA, 23 bp, Artificial Sequence
  primers
acagacagccctcatagttagcg

(19) United States
(12) Patent Application Publication
(10) Pub. No.: US 2005/0059801 A1
(43) Pub. Date: Mar. 17, 2005

(54) Isolated VSHK-1 receptor polypeptides and methods of use thereof

(76) Inventors: Hamiduddin Khoja, Emeryville, CA (US); Venkatarkrishna Shyamala, Emeryville, CA (US)

Correspondence Address: Chiron Corporation;Intellectual Property - R440, P.O. Box 8097, Emeryville, CA, 94662-8097, US

(21) Appl. No.: 10/698,959

(22) Filed: Oct. 30, 2003

Related U.S. Application Data
(63) Continuation of application No. 09/433,360, filed on Nov. 3, 1999, now abandoned.
(60) Provisional application No. 60/107,112, filed on Nov. 4, 1998.
(60) Provisional application No. 60/114,856, filed on Jan. 6, 1999.

(51) Int. Cl. C12Q 1/68; C07H 21/04; C07K 14/705; C07K 16/28

(52) U.S. Cl. 530/350; 530/388.22; 435/69.1; 435/320.1; 435/325; 536/23.5

(57) ABSTRACT

A new seven transmembrane receptor has been identified, and the amino acid and nucleotide sequence of the receptor are provided. The nucleotide sequence is useful to construct expression cassettes and vectors to produce host cells which are capable of expressing the receptor, its mutants, fragments, or fusions. Such polypeptides are useful for identifying new receptor binding agonists and antagonists.

### CROSS REFERENCE TO RELATED APPLICATIONS

This application claims the benefit of prior U.S. Provisional Application No. 60/107,112, filed Nov. 4, 1998 and of prior U.S. Provisional Application No. 60/114,856, filed Jan. 6, 1999, which applications are incorporated herein by reference.

### FIELD OF THE INVENTION

This invention relates to the fields of molecular biology and pharmaceutical research. More specifically, this invention relates to the identification and recombinant expression of a new seven transmembrane receptor, named VSHK-1, and its use to measure ligand binding, signal transduction and in methods to identify novel receptor agonists and antagonists.

### BACKGROUND OF THE INVENTION

The seven transmembrane receptor family comprises receptors that contain seven helical domains which span the cell membrane. The transmembrane regions are linked by three intracellular and three extracellular loops; in addition, such receptors possess an extracellular amino terminal tail and an intracellular carboxyl terminal tail.

The extra- and intracellular loops contribute to the ligand binding and the signal transduction activity of the receptor. For example, the intracellular domains of the receptor are known to be coupled to guanyl-nucleotide-binding proteins, or G-proteins. G-proteins interconvert between GDP- and GTP-binding forms. Binding of endogenous ligand to the receptor triggers the conversion of the G-protein to its GTP-binding form, which initiates the cascade of reactions to generate the desired biological response. This cascade is called signal transduction. Specifically, signal transduction of the seven transmembrane receptors causes an increase of intracellular Ca²⁺ levels and an activation of phospholipase C. Signal transduction can be measured by observing the levels of inositol triphosphate (IP₃) and diacylglycerol (DAG), which are increased due to phospholipase C activation and cyclic AMP (cAMP).

### SUMMARY OF THE INVENTION

The present invention relates to a new seven transmembrane receptor, referred to as VSHK-1 receptor polypeptide. Accordingly, the invention provides isolated VSHK-1 receptor polypeptides. These receptor polypeptides are useful for identifying -substances that modulate one or more VSHK-1 functions. Such substances include novel VSHK-1 receptor binding agonists and antagonists. Compositions comprising the isolated VSHK-1 receptor polypeptides are further provided.

The invention further provides isolated VSHK-1 polynucleotides comprising nucleotide sequences encoding native VSHK-1 receptor polypeptides. These polynucleotides are useful for producing VSHK-1 receptor polypeptides. VSHK-1 polynucleotides are further useful for detecting hybridizing polynucleotides, and can therefore be used to detect the presence of and/or measure a level of VSHK-1 mRNA in a biological sample, as well as to detect related polynucleotides. They are further useful in assays to identify substances which modulate a level of VSHK-1 mRNA. Recombinant vectors and host cells comprising the isolated polynucleotides are further provided.

The invention further provides an antibody which specifically binds VSHK-1 receptor polypeptides. Such antibodies are useful in assays to detect the presence of VSHK-1 receptor polypeptides.

Yet another object of the invention is to provide a method for determining (i.e., detecting or measuring) ligand binding to VSHK-1 receptor polypeptides to identify receptor binding agonists or antagonists. The method comprises contacting a VSHK-1 receptor polypeptide with a substance to be tested under conditions that permit formation of a ligand/receptor complex; and detecting the complex formed between a VSHK-1 receptor polypeptide and the substance.

A further object of the invention is to provide methods for determining (i.e., detecting and/or measuring a level of) VSHK-1 receptor polypeptide signal transduction activity to identify receptor binding agonists or antagonists. The methods generally comprise providing a cell expressing a VSHK-1 receptor polypeptide (or cell membrane preparation from such a cell); exposing the expressed VSHK-1 receptor polypeptide to a ligand under conditions that permit formation of a ligand/receptor complex; and measuring VSHK-1 receptor polypeptide signal transduction activity.

Another object of the invention is to provide methods for detecting VSHK-1 polynucleotides in a biological sample. The methods generally comprise hybridizing a sample comprising polynucleotides to a VSHK-1 polynucleotide of the invention under conditions which favor nucleic acid hybridization, and detecting hybridization, if any.

A further object of the invention is to provide methods for detecting VSHK-1 receptor polypeptides in a biological sample. The methods generally comprise contacting a biological sample with a VSHK-1-specific antibody, and detecting antibody binding.

In another embodiment, the present invention provides both compositions and methods for treating or ameliorating VSHK-1 receptor-mediated disorder and/or accompanying biological and physical manifestations. The compositions for treatment or amelioration include: polynucleotides comprising the sequence capable of hybridizing to a VSHK-1 polynucleotide or complement thereof, including antisense, ribozyme and gene therapy nucleic acid constructs; VSHK-1 receptor polypeptides; and antibodies capable of specifically binding VSHK-1 receptor polypeptides.

These and other objects, aspects, advantages, and features of the invention will become apparent to those persons skilled in the art upon reading the details of the invention as more fully described below.

### BRIEF DESCRIPTION OF THE DRAWINGS

FIG. 1 depicts the nucleotide and amino acid sequence of VSHK-1 (SEQ ID NO:1 and NO:2, respectively).

FIG. 2 depicts the results of Northern blot analysis-of various human tissues using VSHK-1 polynucleotide encoding amino acids 114 to 223 of SEQ ID NO:2.

FIG. 3 depicts an amino acid sequence alignment of a VSHK-1 receptor polypeptide amino acid sequence as set forth in SEQ ID NO:1 with amino acid sequences of human chemokine receptors CCR6, CCR7, and CXCR2, using the ClustalW program with default parameters.

FIG. 4 depicts the results of FACS analysis of HEK293 cells expressing epitope-tagged VSHK-1. Epitope-specific antibody binding to mock-transfected cells (dashed lines) and to cells transfected with epitope-tagged VSHK-1-expression construct (solid lines) is shown.

### MODES OF CARRYING OUT THE INVENTION

The present invention provides the amino acid and nucleotide sequence of a VSHK-1 receptor. With the disclosed nucleotide sequences, nucleic acid probe assays and expression cassettes and vectors for VSHK-1 receptor polypeptides can be produced. The expression vectors can be introduced, transiently or stably, into host cells to produce VSHK-1 receptor polypeptides. The purified receptor polypeptides can be used to produce antibodies to VSHK-1 receptor polypeptides. Also, the host cells or extracts can be utilized for biological assays to isolate receptor binding agonists or antagonists.

Before the present proteins, polynucleotides, antibodies, assays and methods of the invention are disclosed and described, it is to be understood that this invention is not limited to particular sequences, antibodies, assays and the like as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are incorporated herein by reference to disclose and describe methods and/or materials in connection with which the publications are cited. Nothing herein is to be construed as an admission that the present invention is not entitled to antedate such publication by virtue of prior invention. Further, the dates of publication provided are subject to change if it is found that the actual date of publication is different from that provided here.

Definitions

For the purposes of the present application, singular forms “a”, “and”, and “the” include plural referents unless the context clearly indicates otherwise. Thus, for example, reference to “a polypeptide”includes large numbers of polypeptides, reference to “an agent” includes large numbers of agents and mixtures thereof, reference to “the method” includes one or more methods or steps of the type described herein.

The term “polypeptide” refers to a polymer of amino acids and does not refer to a specific length of the product; thus, peptides, oligopeptides, and proteins are included within the definition of polypeptide. The term encompasses polypeptides comprising chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. This term encompasses polypeptides with post-translational modifications of the polypeptide, for example, glycosylations, acetylations, phosphorylations and the like. Included within the definition are, for example, polypeptides containing one or more analogs of an amino acid (including, for example, unnatural amino acids, non-coded amino acids, etc.), polypeptides with substituted linkages, as well as other modifications known in the art, both naturally occurring and non-naturally occurring.

A polypeptide may have a certain “percent sequence identity” when its amino acid sequence is compared with the amino acid sequence of another polypeptide. Similarly, a polynucleotide may have a certain “percent sequence identity” when its nucleotide sequence is compared with the nucleotide sequence of another polynucleotide. To determine sequence identity, sequences can be aligned using the methods and computer programs, including BLAST, available over the world wide web at http://ww.ncbi.nlm.nib.gov/BLAST/. Another alignment algorithm is Fasta, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences.

The FastDB algorithm -may be used to determine sequence identity. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

| | |
|---|---|
| Mismatch Penalty: | 1.00; |
| Gap Penalty: | 1.00; |
| Gap Size Penalty: | 0.33; and |
| Joining Penalty: | 30.0. |

One parameter for determining percent sequence identity is the “percentage of the alignment region length” where the strongest alignment is found.

The percentage of the alignment region length is calculated by counting the number of residues of the individual sequence found in the region of strongest-alignment. This number is divided by the total residue length of the target or query polynucleotide sequence to find a percentage. An example is shown below:

| Target sequence: | GCGCGAAATACTCACTCGAGG |
|---|---|
| | \| \|\| \| \|\| \|\| \|\| \| |
| Query sequence: | TATAGCCCTAC.CACTAGAGTCC |
| | 1 5 10 15 |

The region of alignment begins at residue 9 and ends at amino acid 19. The total length of the target sequence is 20 residues. The percent of the alignment region length is 11 divided by 20 or 55%, for example.

Percent sequence identity is calculated by counting the number of residue matches between the target and query polynucleotide sequence and dividing total number of matches by the number of residues of the target or query sequence found in the region of strongest alignment. For the example above, the percent identity would be 10 matches divided by 11 residues, or approximately, 90.9%.

The percent of the alignment region length, typically, is at least about 55% of total length of the sequence; more typically, at least about 58%; even more typically; at least about 60% of the total residue length of the sequence. Usually, percent length of the alignment region can be as much as about 62%; more usually, as much as about 64%; even more usually, as much as about 66%.

The terms “polynucleotide” and “nucleic acid”, used interchangeably herein, refer to a polymeric forms of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

For hybridization probes, it may be desirable to use nucleic acid analogs, in order to improve the stability and binding affinity. A number of modifications have been described that alter the chemistry of the phosphodiester backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

By “antisense polynucleotide” is mean a polynucleotide having a nucleotide sequence complementary to a given polynucleotide sequence (e.g, a polynucleotide sequence encoding a VSHK-1 receptor polypeptide) including polynucleotide sequences associated with the transcription or translation of the given polynucleotide sequence (e.g., a promoter of a polynucleotide encoding VSHK-1 receptor polypeptide), where the antisense polynucleotide is capable of hybridizing to a VSHK-1 receptor polypeptide-encoding polynucleotide sequence. Of particular interest are antisense polynucleotides capable of inhibiting transcription and/or translation of a VSHK-1 polynucleotide either in vitro or in vivo.

As used herein, the term “native VSHK-1 receptor” refers to the polypeptides found in nature including allelic variants and polymorphisms. An example is a native human VSHK-1 receptor polypeptide, which exhibits substantial amino acid sequence identity to SEQ ID NO:2.

“Native VSHK-1 receptor activity” refers to the biological and biochemical activities that native polypeptides exhibit, which includes, ligand binding, immunological, signal transduction, and therapeutic activities.

“Signal transduction activity” occurs when a ligand binds to the VSHK-1 receptor and triggers a biological response in a cell or cell extract. The biological response is the result of a cascade of biochemical reactions. Measurement of any one of these reactions can indicate that a biological response was triggered. For example, VSHK-1 receptor is a G-coupled protein which, when proper signal transduction activity occurs, triggers an increase of intracellular Ca²⁺, IP₃, and DAG levels. An assay for increased levels of free cytosolic Ca²⁺ is described in Sakurai et al., EP 480 381, and Adachi et al. (1992) FEBS Lett 311:179-183. Intracellular IP₃ concentrations can be measured according to Sakurai et al., EP 480 381 and Amersham's inositol 1,4,5-trisphosphate assay system (Arlington Heights, Ill., U.S.A.). These assays are effective for determining VSHK-1 receptor signal transduction activity whether the receptor is naturally expressed by the cell or expressed by a heterologous cell type by recombinant techniques. Proper signal transduction activity depends not only on receptor/ligand binding but also depends on the presence of the particular intracellular proteins. Thus, while a number of cells are capable, via recombinant techniques, of expressing VSHK-1 receptor polypeptides, no biological response will be detected despite proper receptor/ligand binding if the host cell does not produce the particular intracellular proteins. Heterologous host cells, COS and Chinese Hamster Ovary (CHO) cells, for instance, can produce a biological response if altered to produce the receptor by recombinant techniques. Signal transduction activity also can be detected in cells that are known to naturally express the VSHK-1 receptor in humans, such as heart cells.

As used herein the term “isolated” is meant to describe a polynucleotide, a polypeptide, an antibody, or a host cell that is in an environment different from that in which the polynucleotide, the polypeptide, the antibody, or the host cell naturally occurs. As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the cormposition, more preferably at least about 95% or even 99% by weight.

Two elements are “heterologous” if they are not associated together in nature. For example, a mouse promoter inserted into a human-cell is heterologous to the cell. As another example, a human endothelin promoter is heterologous to a VSHK-1 coding sequence, since the endothelin promoter is not associated with the VSHK-1 receptor-coding sequence in nature.

“Operably linked” refers to a juxtaposition wherein the components so described are in a relationship permitting them to function in their intended manner. For instance, a promoter is operably linked to a coding sequence if the promoter affects its transcription or expression.

“Regulatory sequences” refer to those sequences normally associated with (for example within 50 kb) of the coding region of a locus which affect the expression of the gene (including transcription of the gene, and translation, splicing, stability, or the like of the messenger RNA). Regulatory sequences include, inter alia, promoters, enhancers, splice sites and polyadenylation sites.

A “promoter” is a DNA sequence that initiates and regulates the transcription of a coding sequence when the promoter is operably linked to the coding sequence. A promoter is “heterologous” to the coding sequence when the promoter is not operably linked to the coding sequence in nature. A “native” promoter is operably linked to the coding sequence in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. With certain origins of replication, an expression vector can be reproduced at a high copy number in the presence of the appropriate proteins within the cell. Examples of origins are the 2 μ and autonomously replicating sequences, which are effective in yeast; and the viral T-antigen, effective in COS-7 cells.

A “host cell”, as used herein, denotes microorganisms or eukaryotic cells or cell lines cultured as unicellular entities which can be, or have been, used as recipients for recombinant vectors or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

Host cells capable of producing VSHK-1 receptor polypeptides are cultured “under conditions inducing expression.” Such conditions allow transcription and translation of the DNA molecule encoding the VSHK-1 receptor polypeptide. These conditions include cultivation temperature, oxygen concentration, media composition, pH, etc. An example is when the trp promoter is utilized in the expression vector, then the media will lack tryptophan to trigger the promoter and induce expression. The exact conditions will vary from host cell to host cell and from expression vector to expression vector.

The term “immunologically active” defines the capability of the natural, recombinant or synthetic VSHK-1 receptor polypeptide, or any oligopeptide thereof, to induce a specific immune response in appropriate animals or cells and to bind with specific antibodies. As used herein, “antigenic amino acid sequence” means an amino acid sequence that, either alone or in association with a carrier molecule, can elicit an antibody response in a mammal.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, altered antibodies, univalent antibodies, the Fab proteins, and single domain antibodies.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “VSHK-1 receptor-mediated disorder” refers to a disease state or malady which is caused or exacerbated by an activity of VSHK-1 receptor polypeptides. A “VSHK-1 mediated disorder” refers to a disease state or malady which is caused, exacerbated, modulated, or ameliorated by a biological activity of VSHK-1. Examples include glomerulonephritis, asthma, inflammatory bowel disease, allogeneic transplantation rejection, rheumatoid arthritis, inflammation, tissue damage associated with reperfusion injury and chemical pneumonitis, viral infection, angiogenesis, cancer, or hyperproliferation of cells. Another VSHK-1 mediated disorder is the tissue damage associated with reperfusion injury and chemical pneumonitis.

A “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids and tissue samples.

Isolated VSHK-1 Receptor Polypeptides

The present invention provides isolated VHSK-1 receptor polypeptides. VSHK-1 receptor polypeptides can be used to generate antibodies which specifically bind to VSHK-1 receptor polypeptides. VSHK-1 receptor polypeptides are also useful in assay methods to identify agents which modulate VSHK-1 receptor signal transduction activity, in assays to identify proteins that interact with VSHK-1 receptor polypeptides, and in assay methods to identify ligands of VSHK-1 receptor polypeptides.

In certain embodiments of interest, a VSHK-1 receptor polypeptide is present in a composition that is substantially free of the constituents that are present in its naturally occurring environment. For example, a VSHK-1 receptor polypeptide comprising composition according to the subject invention in this embodiment will be substantially, if not completely, free of those other biological constituents, such as proteins, carbohydrates, lipids, etc., with which it is present in its natural environment. As such, protein compositions of these embodiments will necessarily differ from those that are prepared by purifying the protein from a naturally occurring source, where at least trace amounts of the protein's constituents will still be present in the composition prepared from the naturally occurring source.

A VSHK-1 receptor polypeptide of the subject invention may also be present as an isolate, by which is meant that the VSHK-1 receptor polypeptide is substantially free of both non-VSHK-1 receptor proteins and other naturally occurring biologic molecules, such as oligosaccharides, polynucleotides and fragments thereof, and the like, where-substantially free in this instance means that less than 70%, usually less than 60% and more usually less than 50% of the composition containing the isolated VSHK-1 receptor polypep tide is a non-VSHK-1 receptor naturally occurring biological molecule. In certain embodiments, the VSHK-1 receptor polypeptide is present in substantially pure form, where by substantially pure form is meant at least 95%, usually at least 97% and more usually at least 99% pure.

The term “VSHK-1 receptor polypeptide” encompasses VSHK-1 receptor polypeptides from a variety of eukaryotic species, including, but not limited to, mammalian species, such as rat, mouse, and human; insect species; reptiles; yeast; nematodes; and amphibians.

As used herein, “VSHK-1 receptor polypeptide” refers to an amino acid sequence of a recombinant or non-recombinant polypeptide having an amino acid sequence of i) a native VSHK-1 receptor polypeptide, ii) a fragment of a VSHK-1 receptor polypeptide, iii) polypeptide analogs of a VSHK-1 receptor polypeptide, iv) variants of a VSHK-1 receptor polypeptide; v) an immunologically active fragment of a VSHK-1 receptor polypeptide; vi) isoforms of a VSHK-1 receptor polypeptide; and vii) fusion proteins comprising a VSHK-1 receptor polypeptide. VSHK-1 receptor polypeptides of the invention can be obtained from a biological sample, or from any source whether natural, synthetic, semi-synthetic or recombinant.

“VSHK-1 receptor polypeptides” include mutants, fragments, and fusions as well as native VSHK-1 receptors. Mutants of the native VSHK-1 receptor polypeptides include additions, substitution, or deletions of native VSHK-1 receptor polypeptides. Fragments may possess the same amino acid sequence as native or mutant VSHK-1 receptor polypeptides except fragments lack the amino and/or carboxyl terminal sequences of the native or mutant VSHK-1 receptor polypeptides. Fusions are mutants, fragments, or native VSHK-1 receptor polypeptides that include amino and/or carboxyl terminal amino acid extensions. The number or type of the amino acid substitutions, additions, or deletions is not critical, nor is the length or number of the amino acid deletions, or amino acid extensions that are incorporated in the VSHK-1 receptor polypeptides. However, all of these polypeptides will exhibit at least 20% of at least one of the native VSHK-1 receptor polypeptide activities. More typically, the polypeptides exhibit at least 40%, even more typically the polypeptides exhibit at least 60% of at least one of the native VSHK-1 receptor activities. All these polypeptides will retain at least about 50% amino acid identity with SEQ ID NO:2; more typically, at least 60%; even more typically, at least 80%. Preferably, these polypeptides will retain at least 85% amino acid sequence identity with SEQ ID NO:2; more preferably, at least 90%; even more preferably, at least 91%, 92%, or 93% sequence identity; even more preferably at least 94%, 95%, or 96% sequence identity; even more preferably, at least 97%, 98% or 99% sequence identity. The percent amino acid sequence identity is calculated using the ClustalW program, using default parameters according to software specifications,. which include: open gap penalty=10; extended gap penalty=0.1; similarity matrix=BLOSUM. In addition, VSHK-1 receptor polypeptides can include up to 50 amino acid changes as compared to a native VSHK-1 receptor polypeptide sequence; generally, up to 35 amino acids; more generally, up to 30, 25, or 20 amino acid changes; even more generally, up to 18, 15, or 12 amino acid changes; even more generally, up to 10, 9, 8, 7, 6, 5, 4, 3, 2, or 1 amino acid changes.

“VSHK-1 receptor polypeptide” refers to the amino acid sequences of isolated VSHK-1 receptor polypeptide obtained from a prokaryotic or eukaryotic organism, and is meant to include all naturally-occurring allelic variants, and is not meant to limit the amino acid sequence to the complete, native amino acid sequence associated with the recited protein molecule. The term “VSHK-1 receptor polypeptide” encompasses an amino acid sequence encoded by an open reading frame (ORF) of the VSHK-1 polynucleotides described herein, including the full-length native polypeptide and fragments thereof, particularly biologically active fragments and/or fragments corresponding to structural or functional domains, e.g., a transmembrane domain, a signal transduction domain or region, a ligand-binding domain, a G-protein-binding domain, etc.; and including fusions of the subject polypeptides to other proteins or parts thereof.

Those skilled in the art will appreciate that changes can be made to VSHK-1 receptor polypeptide sequences, including the sequences depicted in SEQ ID NO:2 (VSHK-1) without substantially affecting a function of the VSHK-1 receptor polypeptide. Thus, the term “VSHK-1 receptor polypeptide” encompasses polypeptides with conservative amino acid substitutions compared with the sequences depicted in SEQ ID NO:2. Examples of conservative amino acid substitutions include Ser/Thr; Ala/Val; Leu/Ile; Asp/Glu; and Phe/Tyr/Trp. Clearly, other amino acid substitutions, deletions, and insertions can be made to the polypeptide without affecting one or more functions of the polypeptide.

In many preferred embodiments, VSHK-1 receptor polypeptide is present in its naturally glycosylated state, i.e. it will have the same glycosylation pattern as that found in naturally occurring VSHK-1 receptor polypeptide such that it is a glycoprotein. In other embodiments, the proteins are non-naturally glycosylated. By non-naturally glycosylated is meant that the protein has a glycosylation pattern, if present, which is not the same as the glycosylation pattern found in the corresponding naturally occurring protein. Non-naturally glycosylated VSHK-1 receptor polypeptides of this embodiment include non-glycosylated VSHK-1 receptor polypeptide, i.e. proteins having no covalently bound glycosyl groups.

A subset of mutants, or “muteins”, is a group of polypeptides with the non-disulfide bond participating cysteines substituted with a neutral amino acid, generally, with serines. These mutants may be stable over a broader temperature range than native VSHK-1 receptor polypeptides. Preferably, cysteines in the N-terminal loop and the third extracellular region are conserved if disulfide bonding is desired between these two regions. These residues include Cys 112 and Cys 184. However, cysteines in other regions can be substituted or deleted. In addition, the third intracellular loop fragment of native VSHK-1 receptor polypeptides is basic due to the number of residues that are positively charged at physiological pH. Changes to the third intracellular loop can include those that permit the loop fragment to remain positively charged at physiological pH. To retain an overall net positive charge at the desired pH, a number of positively charged amino acids, such as histidine, lysine, arginine, asparagine, and glutamine, can be substituted or inserted into the native VSHK-1 receptor amino acid sequence.

Further, the N-terminal loop of native VSHK-1 receptor polypeptides are acidic, containing a number of residues that are negatively charged at physiological pH. Thus, substitutions, additions, or deletions can be made that permit the loop fragment to retain a negative charge at the desired pH. Such naturally occurring residues, including aspartic acid and glutamic acid, retain a negative charge under physiological pH and can be substituted or inserted into mutants of the N-terminal loop to retain acidic characteristics.

The N-terminal loop can be used as a viral inhibitor which binds to infecting virus to prevent entry into a cell. Typically, the N-terminal loop fragment can be mutated, but will retain its acidic nature and will retain its tyrosine residues. Two potential glycosylation sites in the N-terminal loop can be conserved, deleted, or altered, depending on whether glycosylation is desired.

Preferably, the arginine in the seventh transmembrane domain is conserved or substituted with another positively charged amino acid, such as histidine, lysine, asparagine, or glutamine.

Other motifs that can be conserved in VSHK-1 receptor polypeptides are located in transmembrane regions as follows:

| | |
|---|---|
| TM1: | GNXXV |
| TM2: | IXNLAXAADL |
| TM3: | LXXISXDRY |
| TM4: | WXXAXXXXXP |
| TM5: | FXXPXXXMXXXY |
| TM6: | KXXXXXXXXFXXXXXPY |
| TM7: | SXXNPXXY. |

The one-letter amino acid abbreviations are used to describe the above-listed motifs, with X indicating any amino acid.

Those skilled in the art, given the guidance provided in the instant specification, can readily determine whether a given function of a VSHK-1 receptor polypeptide is preserved. One such function is signal transduction by a VSHK-1 receptor polypeptide of the invention. Yet another function is binding to other protein(s), e.g., one or more G-proteins. Still another function is ligand binding.

Whether a VSHK-1 receptor polypeptide functions in signal transduction and/or ligand binding and/or G-protein binding is readily determined, using any known assay, including the signal transduction assays described herein.

The term “VSHK-1 receptor polypeptide” encompasses a polypeptide comprising 8 or more contiguous amino acids of the sequence depicted in SEQ ID NO:2. Thus, the term “VSHK-1 receptor polypeptide” encompasses a polypeptide comprising at least about 8, 10, 15, 20, 25, 50, 75, 100, 200, 300, or 325 contiguous amino acids of the sequence set forth in SEQ ID NO:2. In some embodiments, a VSHK-1-polypeptide has the entire sequence as shown in SEQ ID NO:2. The term “VSHK-1 receptor polypeptide” also encompasses a polypeptide comprising at least about 8, 10, 15, 20, 25, 50, 75, or 80 contiguous amino acids of amino acids 1-80 of the sequence set forth in SEQ ID NO:2. The term “VSHK-1 receptor polypeptide” also encompasses a polypeptide comprising at least about 8, 10, 15, 20, 25, 50, 75, 100, 125, 150; or 162 contiguous amino acids of amino acids 189-350 of the sequence set forth in SEQ ID NO:2.

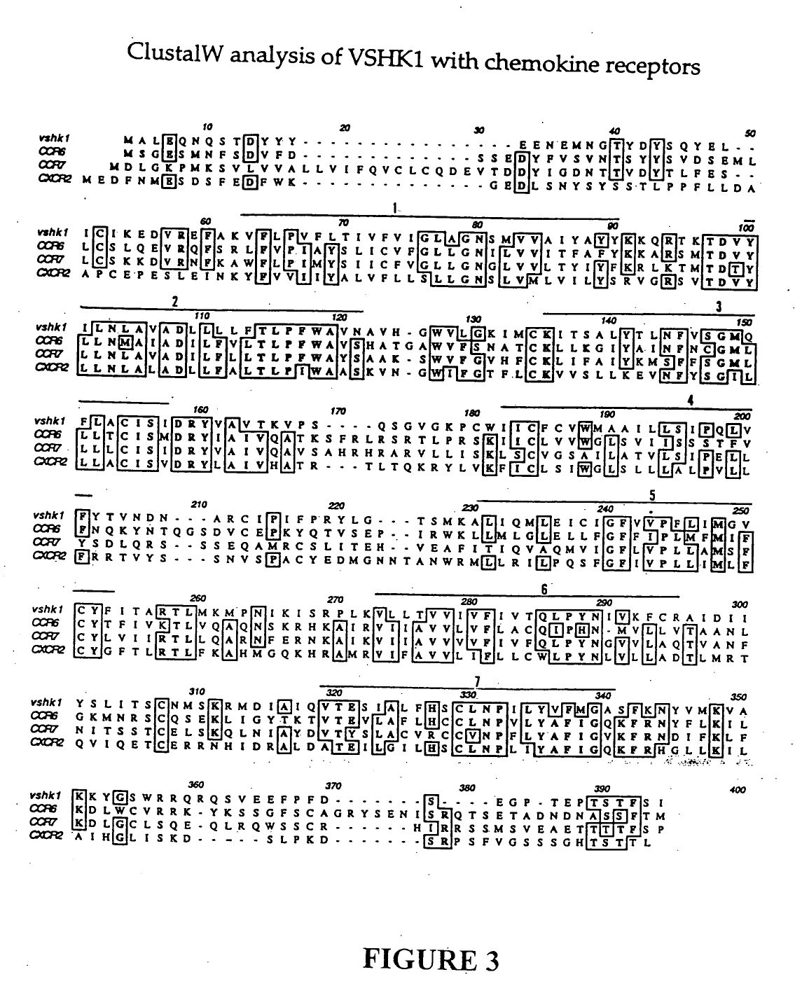

VSHK-1 receptor polypeptide having the sequence set forth in SEQ ID NO:2 has some sequence similarity to chemokine receptors. Chemokine receptors play pivotal roles-in leukocyte migration, and in entry of human immunodeficiency virus into cells. See, for example, Pelchen-Matthews et al. (1999) Immunol Rev. 168:33-49; Wells et al. (1999) Immunol. Lett. 65:35-40; and Berger et al. (1999) Ann. Rev. Immunol. 17:657-700. FIG. 3shows a VSHK-1 receptor polypeptide having the sequence set forth in SEQ ID NO:2 aligned with chemokine receptors CCR6, CCR7, and CXCR2. CCR6 and CCR7 share 32% and 37% amino acid sequence identity, respectively, with the amino acid sequence set forth in SEQ ID NO:2. The alignment was performed using the ClustalW program, using default parameters provided by the software developer. The ClustalW program is described in Thompson et al. (1994) Nucl. Acids Res. 22:4673-4680.

Accordingly, also encompassed by the term “VSHK-1 receptor polypeptide” is a polypeptide sharing at least about 40%, more preferably at least about 50%, more preferably at least about 60%, more preferably at least about 70%, more preferably at least about 80%, more preferably at least about 85%, even more preferably at least about 90% or more amino acid sequence identity with the sequence depicted in SEQ ID NO:2, as calculated using the ClustalW program with default parameters.

On the basis of similarity with other chemokine receptors, a VSHK-1 receptor polypeptide may exhibit one or more of the following biological activities: (1) Mediation of chemotaxis of neutrophils, lymphocytes, tumor-infiltrating, lymphocytes, hemopoietic progenitors, monocytes, natural killer cells. Assays for chemotaxis relating to neutrophils are described in Walz et al. (1987) Biochem. Biophys. Res. Commun. 149:755; Yoshimura et al. (1987) Proc. Natl. Acad. Sci. USA 84:9233; and Schroder et al. (1987) J. Immunol. 139:3474; lymphocytes, Larsen et al., Science 243: 1464: (1989); Carr et al., Proc. Natl. Acad. Sci. USA 91: 3652 (1994); tumor-infiltrating lymphocytes, Liao et al., J. Exp. Med. 182: 1301 (1995); hemopoietic progentors, Aiuti et al., J. Exp. Med. 185: 111 (1997); monocytes, Valente et al., Biochem. 27: 4162 (1988); natural killer cells, Loetscher et al., J. Immunol. 156: 322 (1996), and Allavena et al. (1994) Eur. J Immunol. 24:3233; (2) involvement in angiogenesis or cell proliferation. The assays for such activities is described in Maione et al., Science 247: 77 (1990); (3) involvement in glycosaminoglycan production. A method for detecting this activity is described in Castor et al. (1983) Proc. Natl. Acad. Sci. USA 80:765; (4) involvement in histamine release from basophils. The assay for release is described in Dahinden et al. (1989) J. Exp. Med. 170: 1787; and White et al.(1989) Immunol. Lett. 22:151; (5) Heparin binding as described in Luster et al. (1995) J. Exp. Med. 182:219; (6) involvement in the inflammatory response of viruses. This activity can be assayed as described in Bleul et al. (1996) Nature 382:829; and Oberlin et al. (1996) Nature 382:833; (7) promotion of exocytosis of monocytes. The assay for such activity is described in Uguccioni et al. (1995) Eur. J. Immunol. 25: 64; (8) involvement in hemapoietic stem cell proliferation. The method for testing for such activity is reported in Graham et al. (1990) Nature 344: 442.

Also encompassed by the term “VSHK-1 receptor polypeptide” are specific fragments of VSHK-1. Specific fragments of VSHK-1 receptor polypeptides provided by the present invention include (1) fragments of the N-terminal extracellular tail of native VSHK-1 comprising amino acids 1 to 41, amino acids 10 to 41, amino acids 30 to 41 of SEQ ID NO:2; (2) fragments of the second extracellular loop of native VSHK-1 receptor polypeptide, comprising amino acids 100 to 112, amino acids 105 to 112, amino acids 97 to 115 of SEQ ID NO:2; (3) fragments of the third extracellular loop of native VSHK-1 receptor polypeptide, comprising amino acids 178 to 198, amino acids 180 to 190, amino acids 175 to 201 of SEQ ID NO:2; (4) fragments of the fourth extracellular loop of native VSHK-1 receptor polypeptide, comprising amino acids 264 to 282, amino acids 267 to 279, amino acids 257 to 295 of SEQ ID NO:2; (5) the first transmembrane domain of native VSHK-1 receptor polypeptide, comprising amino acids 42 to 67, amino acids 39 to 70, amino acids 58 to 62 of SEQ ID NO:2; (6) the second transmembrane domain of native VSHK-1 receptor polypeptide, comprising amino acids 74 to 99, amino acids 71 to 102, amino acids 80 to 88 of SEQ ID NO:2; (7) the third transmembrane domain of native VSHK-1 receptor polypeptide, comprising 1 13 to 138, amino acids 110 to 141, amino acids 130 to 138 of SEQ ID NO:2; (8) the fourth transmembrane domain of native VSHK-1 receptor polypeptide, comprising amino acids 152 to 177, amino acids 149 to 180, amino acids 160 to 172 of SEQ ID NO:2; (9).the fifth transmembrane domain of native VSHK-1 receptor polypeptide, comprising amino acids 198 to 224, amino acids 195 to 227, amino acids 209 to 220 of SEQ ID NO:2; (10) the sixth transmembrane domain of native VSHK-1 receptor polypeptide, comprising amino acids 238 to 263, amino acids 235 to 266, amino acids 240 to 256 of SEQ ID NO:2; (11) the seventh transmembrane domain of native VSHK-1 receptor polypeptide, comprising amino acids 283 to 308, amino acids 280 to 311, amino acids 296 to 303 of SEQ ID NO:2; (12) the C-terminal intracellular tail of native VSHK-1 receptor polypeptide, comprising amino acids 309 to 350, amino acids 330 to 350, amino acids 340 to 350 of SEQ ID NO:2; (13) the first intracellular loop of native VSHK-1 receptor polypeptide, comprising amino acids 68 to 73, amino acids 68 to 76, amino acids 65 to 76 of SEQ ID NO:2; (14) the second intracellular loop of native VSHK-1 receptor polypeptide, comprising amino acids 139 to 151, amino acids 139 to 154, amino acids 136 to 154 of SEQ ID NO:2; and (15) the third intracellular loop of native VSHK-1 receptor polypeptide, comprising amino acids 225 to 237, amino acids 222 to 237, amino acids 222 to 240 of SEQ ID NO:2. Also included are polypeptides comprising one or more of the foregoing fragments; fusion proteins including one or more of the foregoing fragments; any of the foregoing fragments comprising conservative amino acid changes, and polypeptides comprising such fragments.

VSHK-1 receptor polypeptides can be used in methods to detect factors, such as proteins, that bind to a VSHK-1 receptor polypeptide. Accordingly, the invention provides methods of identifying proteins that bind to a VSHK-1 receptor polypeptide. The methods generally comprise contacting a test substance with a VSHK-1 receptor polypeptide under conditions that permit formation of a protein-protein complex; and detecting the presence of any complexes formed. Any known method for identifying interacting proteins can be used; including, but not limited to, a yeast two-hybrid screen, also known as protein interactive trapping; and interaction cloning. These methods have been amply described in the literature and need not be described in detail herein. Publications describing these methods include, for example, Current Protocols in Molecular Biology, (F. M. Ausubel, et al., Eds. 1987, and updates); Short Protocols in Molecular Biology (F. M. Ausubel et al., Eds. 1999), Chapter 19; Blanar and Ruttner (1992) Science 256:1014-1018; and McAlister-Henn et al. (1999) Methods 19:330-337, and references cited therein describing protein interactive trapping. These methods can be used, for example, to identify a G-protein which binds to a VSHK-1 receptor polypeptide of the invention.

Also included in the term “VSHK-1 receptor polypeptide” are antigenic epitopes of a VSHK-1 receptor polypeptide. Extracellular loops are exposed on the cell surface and would therefore be more likely to contain antigenic epitopes, including the above-described peptide fragments. Those skilled in the art can readily determine which peptide fragments are antigenic epitopes. As a non-limiting example of how one can determine which region(s) of a protein are likely to be exposed on the surface (i.e., hydrophilic domains), and therefore potentially antigenic, one can analyze the amino acid sequence using Kyte-Doolittle hydropathicity analysis and/or Hopp-Woods hydrophilicity analysis. Kyte and Doolittle (1982) J. Mol. Biol. 157:105; and Hopp and Woods (1981) Proc. Natl. Acad. Sci. USA 78:3824.

Production of VSHK-1 Receptor Polypeptides

VSHK-1 receptor polypeptides can be isolated from a biological source, can be produced synthetically, or can be produced recombinantly, i.e., a VSHK-1 receptor-coding region can be inserted into an expression vector, and the VSHK-1 receptor coding region transcribed and translated.

VSHK-1 receptor polypeptides can be isolated from biological sources, using standard methods of protein purification known in the art. VSHK-1 receptor can also be isolated from a biological source by affinity chromatography, using a VSHK-1 receptor-specific antibody, using standard methods known in the art.

One may employ solid phase peptide synthesis techniques, where such techniques are known to-those of skill in the art. See Jones, The Chemical Synthesis of Peptides (Clarendon Press, Oxford)(1994). Generally, in such methods a peptide is produced through the sequential additional of activated monomeric units to a solid phase-bound growing peptide chain.

For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to the subject gene, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the gene. After introduction of the DNA, the cells containing the construct may be selected by means of a selectable marker, the cells expanded and then used for expression.

The polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, or 293 cells, may be used as the expression host cells. In some situations, it is desirable to express the gene in eukaryotic cells, where the protein will benefit from native folding and post-translational modifications. Small peptides can also be synthesized in the laboratory. Polypeptides that are subsets of the complete amino acid sequence may be used to identify and investigate parts of the protein important for function, or to raise antibodies directed against these regions.

The purified VSHK-1 receptor polypeptides are useful for signal transduction assays and ligand/receptor binding assays. The purified polypeptides can also be utilized to produce VSHK-1 receptor polypeptide-specific antibodies.

For ligand/receptor binding studies, the crude cell membrane fractions can be utilized. These membrane extracts can be isolated from cells which expressed VSHK-1 receptor polypeptides by lysing the cells and separating the cell membrane fraction from the intracellular fractions by centrifugation. See Adachi et al. (1992) FEBS Lett. 311: 179-183 for an ligand binding assay procedure using cell membranes.

Once the polypeptide has been dissociated from the cell membrane, the desired VSHK-1 receptor polypeptide can also be affinity purified with specific VSHK-1 receptor antibodies.

With the availability of the protein or fragments thereof in large amounts, by employing an expression host, the protein may be isolated and purified in accordance with conventional ways. A lysate may be prepared of the expression host and the lysate purified using HPLC, exclusion chromatography, gel electrophoresis, affinity chromatography, or other purification technique.

Isolated VSHK1 Polynucleotides

The present invention provides isolated VSHK-1 polynucleotides encoding a VSHK-1 receptor polypeptide. These polynucleotides can be used, when in a recombinant expression vector, to produce the encoded VSHK-1 receptor polypeptide. They are also useful as hybridization probes in methods of detecting VSHK-1 gene expression, specifically transcription. Accordingly, the invention further provides recombinant vectors and host cells comprising VSHK-1 polynucleotides of the invention.

The VSHK-1 polynucleotides of the subject invention are isolated and obtained in substantial purity, generally as other than an intact chromosome. Usually, the DNA will be obtained substantially free of other nucleic acid sequences that do not include a VSHK-1 polynucleotide sequence or fragment thereof, generally being at least about 50%, usually at least about 90% pure and are typically “recombinant”, i.e. flanked by one or more nucleotides with which it is not normally associated on a naturally occurring chromosome.

Novel polynucleotides of the invention comprise a sequence set forth in SEQ ID NO: 1 (VSHK-1 nt), or an identifying sequence thereof. An “identifying sequence” is a contiguous sequence of residues at least about 10 nucleotides (nt) to about 20 nt in length, usually at least about 50 nt to about 100 nt in length, that uniquely identifies the provided sequence.

An expressed sequence tag (EST) was made publicly available through GenBank (GenBank Accession No. H67224). The H67224 EST provides the nucleotide sequence of a 328-nucleotide fragment from human olfactory epithelium.; This sequence is identical to nucleotides 325 to 651 of SEQ ID NO:1. Hillier et al. (1996) Genome Res. 6:807-828.

Encompassed in the term “VSHK-1 polynucleotide” are polynucleotides comprising about 330, 350, 400, 500, 600, 700, 800, 900, 1000, 1500, or 1950 contiguous nucleotides of SEQ ID NO:1, including the entire coding region of SEQ ID NO:1. Also encompassed are polynucleotides comprising nucleotides 1-324 of SEQ ID NO:1, encoding amino acids 1-80 of VSHK-1 having the amino acid sequence set forth in SEQ ID NO:2. Further included are polynucleotides comprising at least about 18, 20, 30, 40, 50, 100, 150, 200, 250, and/or 300 contiguous nucleotides of nucleotides 1-324 of SEQ ID NO:1. Also encompassed are polynucleotides comprising nucleotides 652-1890 of SEQ ID NO:1. Further encompassed are polynucleotides comprising at least about 18, 30, 50, 100, 150, 200, 500, 750, 1000, and/or 1239 contiguous nucleotides of nucleotides 652-1890 of SEQ ID NO:1. Polynucleotides comprising sequences which encode the region of the VSHK-1 receptor protein that interacts with a G-protein are also of interest. Such fragments are often contained within the coding region, and may be about 250 to 500 nucleotides in length, up to the complete coding sequence.

Polynucleotides of the invention also include nucleic acids having sequence similarity or sequence identity to the sequence provided in SEQ ID NO:1. Nucleic acids having sequence similarity are detected by hybridization under low stringency conditions, for example, at 50° C. and 10×SSC (0.9 M NaCl/0.09 M sodium citrate) and remain bound when subjected to washing at 55° C. in 1×SSC. Sequence identity can be determined by hybridization under stringent conditions, for example, at 50° C. or higher and 0.1×SSC (9 mM NaCl/0.9 mM sodium citrate).

Hybridization methods and conditions are well known in the art, see, e.g., U.S. Pat. No. 5,707,829. Nucleic acids that are substantially identical to the provided nucleic acid sequences, e.g. allelic variants, genetically altered versions of the gene, etc., bind to the provided nucleic acid sequence (SEQ ID NO:1) under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

“Hybridization” refers to the association of two nucleic acid sequences to one another by hydrogen bonding. Two sequences will be placed in contact with one another under conditions that favor hydrogen bonding. Factors that affect this bonding include: the type and-volume of solvent; reaction temperature; time of hybridization; agitation; agents to block the non-specific attachment of the liquid phase sequence to the solid support (Denhardt's reagent or BLOTTO);

concentration of the sequences; use of compounds to increase the rate of association of sequences (dextran sulfate or polyethylene glycol); and the stringency of the washing conditions following hybridization. See Sambrook, et al., Molecular Cloning: A Laboratory Manual, 2nd Ed. (1989), Volume 2, chapter 9, pages 9;47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tₘ of the hybrid under study. The temperature and salt conditions can often be determined empirically in preliminary experiments in which samples of genomic DNA immobilized on filters are hybridized to the sequence of interest and then washed under conditions of different stringencies. See Sambrook, et al., supra, at page 9.50.

Variables to consider when performing, for example, a Southern blot are (1) the complexity of the DNA being blotted and (2) the homology between the target and the sequences being detected. The total amount of the polynucleotides to be studied can vary a magnitude of 10, from 0.1 to 1 μg for a plasmid or phage digest to 10⁻⁹ to 10⁻⁸ μg for a single copy gene in highly complex eukaryotic genome. For lower complexity polynucleotides, substantially shorter blotting, hybridization, and exposure times, a smaller amount of starting polynucleotides, and lower specific activity of a target polynucleotide can be used. For example, a single-copy yeast gene can be detected with an exposure time of only 1 hour starting with 1 μg of yeast DNA, blotting for two hours, and hybridizing for 4-8 hours with a target polynucleotide radiolabeled with 10⁸ cpm/μg. For a single-copy mammalian gene a conservative approach would start with 10 μg of DNA, blot overnight, and hybridize overnight in the presence of 10% dextran sulfate using a target polynucleotide radiolabeled with greater than 10⁸ cpm/μg, resulting in an exposure time of ˜24 hours.

Several factors can affect the melting temperature (Tₘ) of a DNA-DNA hybrid between the target and sequence of interest, and consequently, the appropriate conditions for hybridization and washing. In many cases the target is not 100% homologous to the fragment. Other commonly encountered variables include the length and total G+C content of the hybridizing sequences and the ionic strength and formamide content of the hybridization buffer. The effects of all of these factors can be approximated by a single equation:
Tₘ=81+16.6(log 10Ci)+0.4[%G+C)]−0.6(% formamide)−600/n-1.5(% mismatch),
where Ci is the salt concentration (monovalent ions) and n is the length of the hybrid in base pairs (slightly modified from Meinkoth and Wahl (1984) Anal. Biochem. 138:267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e., stringency), it becomes less likely for hybridization to occur between strands that are nonhomologous, and as a result, background decreases. If the labeled probe is-not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a target polynucleotide with 95% to 100% sequence identity to the sequence to be detected, 37° C. for 90% to 95% sequence identity, and 32° C. for 85% to 90% sequence identity. For lower percentage sequence identity, formamide content should be lowered and temperature adjusted accordingly, using the equation above. -If the homology between the target polynucleotide and the sequence to be detected are not known, the simplest approach is to start with both hybridization and wash conditions which are nonstringent. If nonspecific bands or high background are observed after autoradiography, the filter can be washed at high stringency and reexposed. If the time required for exposure makes this approach impractical, several hybridization and/or washing stringencies should be tested in parallel. Stringent conditions include hybridization in a solution of about 5×SSC at 65° C., or at least about 4×SSC at 42° C.; see, for example, U.S. Pat. No. 5,707,829, the disclosure of which is herein incorporated by reference.

Generally, hybridization is performed using at least 18 contiguous nucleotides of SEQ ID NO:1. That is, when at least 18 contiguous nucleotides of the disclosed SEQ ID NO:1 is used as a probe, the probe will preferentially hybridize with a nucleic acid or mRNA comprising the complementary sequence, allowing the identification and retrieval of the nucleic acids of the biological material that uniquely hybridize to the selected probe. Probes of more than 18 nucleotides can be used, e.g. probes of from about 25 nucleotides to about 100 nucleotides, from about 100 nucleotides to about 500 nucleotides, up to the entire coding region can be used, but 18 nucleotides generally represents sufficient sequence for unique identification.

The nucleic acids of the invention also include naturally occurring variants of the nucleotide sequences, e.g. degenerate variants, allelic variants, etc. Variants of the nucleic acids of the invention are identified by hybridization of putative variants with-nucleotide sequences disclosed herein, preferably by hybridization under stringent conditions. For example, by using appropriate wash conditions, variants of the nucleic acids of the invention can be identified where the allelic variant exhibits at most about 25-30% base pair mismatches relative to the selected nucleic acid probe. In general, allelic variants contain 15-25% base pair mismatches, and can contain as few as even 5-15%, or 2-5%, or 1-2% base pair mismatches, as well as a single base-pair mismatch.

Homologs of the VSHK-1 are also provided in the present invention. Such homologs can be identified by any of a number of methods known to those skilled in the art. A fragment of the provided cDNA may be used as a hybridization probe against a cDNA library from the target organism of interest, where low stringency conditions are used. The probe may be a large fragment, or one or more short degenerate primers.

The invention also encompasses homologs corresponding to the nucleic acids of SEQ ID NO:1, where the source of homologous genes can be any related species within the same genus or group. Within a group, homologs have substantial sequence similarity, e.g. at least 75% sequence identity, usually at least 90%, more usually at least 95% between nucleotide sequences, as determined using the BLAST alignment program. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared.

The term “VSHK-1 polynucleotide” encompasses polynucleotides which encode a VSHK-1 polypeptide, a fragment thereof, or a fusion protein thereof, as described above. Thus, in some embodiments, a VSHK-1 polynucleotide comprises a nucleotide-sequence encoding a polypeptide comprising at least about 8, 10, 15, 20, 25, 50, 75, 100, 200, 300, or 325 contiguous amino acids of the sequence set forth in SEQ ID NO:2. In other embodiments, a VSHK-1 polynucleotide comprises a nucleotide sequence encoding the entire polypeptide having the amino acid sequence set forth in SEQ ID NO:2. In other embodiments a VSHK-1 polynucleotide comprises a polynucleotide sequence encoding a polypeptide comprising at least about 8, 10, 15, 20, 25, 50, 75, 100, 125, 150, or 162 contiguous amino acids of amino acids 189-350 of the sequence set forth in SEQ ID NO:2. In still other embodiments, a VSHK-1 polynucleotide comprises a nucleotide sequence which encodes a polypeptide comprising an amino acid sequence sharing at least about 70%, more preferably at least about 80%, even more preferably at least about 90% or more amino acid sequence identity, as determined using the ClustalW program with default parameters, with the sequence depicted in SEQ ID NO:2.

Also encompassed by the term “VSHK-1 polynucleotide” are polynucleotides complementary to a VSHK-1 polynucleotide, as defined above. Further encompassed are VSHK-1 antisense polynucleotides and ribozymes. Various derivatives of the antisense sequence may be prepared, where the phosphates may be modified, where oxygens may be substituted with sulfur and nitrogen, the sugars may be modified, and the like. The antisense sequences may be used by themselves or in conjunction with various toxic moieties, such as metal chelates, sensitizers, ribozymes, and the like. Antisense and/or ribozyme sequences may be used to inhibit spermatogenesis. Antisense polynucleotides, and methods of using such, are described in numerous publications, including, e.g., “Antisense Technology: A Practical Approach” Lichtenstein and Nellen, eds. (1997) IRL Press.

Antisense molecules can be used to down-regulate expression of VSHK-1 genes in cells. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise two or more different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993) supra.) Preferred oligonucleotides are chemically modified from the native phosphodiester structure, in order to increase their intracellular stability and binding affinity. Such modifications have been previously discussed with respect -to the use of probes.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995) Nucl. Acids Res 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995) Appl Biochem Biotechnol 54:43-56.

A VSHK-1 polynucleotide may be a VSHK-1 cDNA. The term “cDNA” as used herein is intended to include all nucleic acids that share the arrangement of sequence elements found in native mature mRNA species, where sequence elements are exons and 3″ and 5″ non-coding regions. Normally mRNA species have contiguous exons, with the intervening introns, when present, removed by nuclear RNA splicing, to create a continuous open reading frame encoding the protein protein

Also encompassed by the term “VSHK-1 polynucleotide” are VSHK-1 genomic sequences. A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, up to about 6 kb, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes-found in introns, contains sequences required for proper tissue and stage specific expression.

The sequence of the 5′ flanking region may be utilized for promoter elements, including enhancer binding sites, that provide for developmental regulation in tissues where VSHK-1 is expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell et al. (1995) Mol. Med. 1:194-205; Mortlock et al. (1996) Genome Res. 6:327-33; and Joulin and Richard-Foy (1995) Eur. J Biochem. 232:620-626.

The regulatory'sequences may be used to identify cis acting sequences required for transcriptional or translational regulation of expression, especially in different tissues or stages of development, and to identify cis acting sequences and trans-acting factors that regulate or mediate expression. Such transcription or translational control regions may be operably linked to one of the subject genes in order to promote expression of wild type or altered VSHK-1 protein, or other proteins of interest in cultured cells, or in embryonic, fetal or adult tissues, and for gene therapy.

The nucleic acid compositions of the subject invention may encode all or a part of the VSHK-1 receptor polypeptides of the invention. Double or single stranded fragments may be obtained of the DNA sequence by chemically synthesizing oligonucleotides in accordance with conventional methods, by restriction enzyme digestion, by PCR amplification, etc. For the most part, DNA fragments will be of at least 18 nt, usually at least 25 nt or 50 nt, and may be at least about 100 nt. Such small DNA fragments are useful as primers for PCR, hybridization screening probes, etc. Larger DNA fragments, i.e. greater than 500 nt are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The polynucleotides of the invention are isolated and obtained in substantial purity, generally as other than an intact chromosome. Usually, the DNA will be obtained substantially free of nucleic acid sequences other than a VSHK-1 polynucleotide, generally being at least about 50%, usually at least about 90% pure and are typically “recombinant”, i.e. flanked by one or more nucleotides with which it is not normally associated on a naturally occurring chromosome.

The DNA may also be used to identify expression of the gene in a biological specimen. The manner in which one probes cells for the presence of particular nucleotide sequences, as genomic DNA or RNA, is well established in the literature and does not require elaboration here. DNA or mRNA is isolated from a cell sample. The mRNA may be amplified by reverse transcriptase-polymerase chain reaction (RT-PCR), using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of VSHK-1 gene expression (i.e., at least transcription) in the sample.

The sequence of genes encoding VSHK-1 receptor, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon. One of skill in the art will recognize that, in general, such mutations will occur outside of regions that affect VSHK-1 signal transduction activity. Other modifications of interest include epitope tagging, e.g. with the FLAG system, HA, etc. For studies of subcellular localization, fusion proteins with green fluorescent proteins (GFP) may be used.

Techniques for in vitro mutagenesis of cloned genes are known. Examples of protocols for site specific mutagenesis may be found in Gustin et al. (1993) Biotechniques 14:22; Barany (1985) Gene 37:111-23; Colicelli et al. (1985) Mol. Gen. Genet. 199:537-9; Prentki et al. (1984) Gene 29:303-13; and Shyamala and Ames (1991) Gene 97:1-6. Methods for site specific mutagenesis can be found in Sambrook et al. Molecular Cloning: A Laboratory Manual, CSH Press 1989, pp. 15.3-15.108; Weiner, et al., (1993) Gene 126:35-41; Sayers et al., (1992) Biotechniques 13:592-6; Jones, et al. (1992) Biotechniques 12:528-30; Barton et al., (1990) Nucleic Acids Res. 18:7349-55; Marotti, et al. (1989) Gene Anal. Tech. 6:67-70; and Zhu (1989) Anal Biochem 177:120-4. Such mutated genes may be used to study structure-function relationships of VSHK-1 polypeptides, or to alter properties of the protein that affect its function or regulation.

VSHK-1 polynucleotides can be prepared in a number of different ways. For example, the nucleic acid may be synthesized using solid phase synthesis techniques, as are known in the art. Oligonucleotide synthesis is also described in Edge et al. (1981) Nature 292:756; Duckworth et al. (1981) Nucleic Acids Res 9:1691 and Beaucage et al. (1981) Tet. Letts 22: 1859. Following preparation of the nucleic acid, the nucleic acid is then ligated to other members of the expression system to produce an expression cassette or system comprising a nucleic acid encoding the subject product in operational combination with transcriptional initiation and termination regions, which provide for expression of the nucleic acid into the subject polypeptide products under suitable conditions.

Recombinant Vectors of the Invention

The present invention further provides recombinant vectors (“constructs”) comprising VSHK-1 polynucleotides of the invention. Recombinant vectors are useful for propagation of the subject VSHK-1 polynucleotides (cloning vectors). They are also useful for effecting expression of a VSHK-1 polynucleotide in a cell (expression vectors). Some vectors accomplish both cloning and expression functions. The choice of appropriate vector is well within the skill of the art. Many such vectors are available commercially.

A variety of host-vector systems may be utilized to propagate and/or express the VSHK-1 polynucleotides of the invention. Such host-vector systems represent vehicles by which coding sequences of interest may be produced and subsequently purified, and also represent cells that may, when transformed or transfected with the appropriate nucleotide coding sequences, produce VSHK-1 receptor polypeptides of the invention. These include, but are not limited to, microorganisms (e.g., E. coli, B. subtilis) transformed with recombinant bacteriophage vectors, plasmid DNA, or cosmid DNA vectors comprising VSHK-1 polynucleotides; yeast (e.g., Saccharomyces, Pichia) transformed with recombinant yeast vectors comprising VSHK-1 polynucleotides); insect cell systems (e.g., Spodoptera frugiperda) infected with recombinant virus expression vectors (e.g., baculovirus vectors, many of which are commercially available, including, for example, pBacPAK8, and BacPAK6) comprising VSHK-1 polynucleotides; plant cell systems; or mammalian cell systems (e.g., COS, CHO, BHK, 293, 3T3) harboring recombinant vectors comprising mammalian promoters (e.g., metallothionein promoter) or promoters from viruses which replicate in mammalian cells (e.g., adenovirus late promoter; vaccinia virus promoter, and the like). Examples of prokaryotic cloning vectors which find use in propagating VSHK-1 polynucleotides of the invention are pBR322, M13 vectors, pUC18, pcDNA, and pUC19. Prokaryotic expression vectors which find use in expressing VSHK-1 polypeptides in prokaryotic cells include pTrc99A, pK223-3, pEZZ18, pRIT2T, and pMC1871. Eukaryotic expression vectors which find use in expressing VSHK-1 polynucleotides and VSHK-1 polypeptides in eukaryotic cells include commercially available vectors such as pSVK3, pSVL, pMSG, pCH110, pMAMneo, pMAMneo-LUC, pPUR, and the like.

Generally, a bacterial host will be transformed to contain the expression system using a vector. A variety of vectors may be employed so long as they introduce the expression system into the host in a manner whereby the product encoded by the expression system can be expressed. Thus, the vector could be one that is capable homologously recombining with a region of the host chromosome such that the expression system becomes integrated into the host chromosome such that expression of the protein encoded thereby can occur. See Thomas and Capecchi (1987) Cell 51:503-512; as well as U.S. Pat. Nos.: 5,631,153; 5,627,059; 5,487,992 and 5,464,764, the disclosure of which is herein incorporated by reference.

Generally, the expression cassette will be a plasmid that provides for expression of the encoded VSHK-1 receptor polypeptide under appropriate conditions, i.e. in a host cell. The expression vector will typically comprise a replicon, which includes the origin of replication and its associated cis-acting control elements. Representative replicons that may be present on the expression vector include: pMB1, p15A, pSC101 and ColE1. Expression vectors generally have convenient restriction sites located near the promoter sequence to provide for the insertion of nucleic acid sequences encoding heterologous proteins. In addition, the expression vector will also typically comprise a marker which provides for detection of the clones that have been transformed with the vector. A variety of markers are known and may be present on the vector, where such markers include those that confer antibiotic resistance, e.g. resistance to ampicillin, tetracycline, chloramphenicol, kanamycin (neomycin), markers that provide for histochemical detection, etc. Specific vectors that may find use in the subject methods include: pBR322, pUC18, pUC19, pcDNA, and the like. Introduction of the nucleic acid encoding the subject peptidic product into the expression vector is accomplished by cutting the expression vector and inserting-the polynucleotide encoding the desired product.

Following preparation of the expression vector comprising the nucleic acid, the expression vector will be introduced into an appropriate host cell for production of the VSHK-1 polypeptide, i.e. a host cell will be transformed with the expression vector. Transformation of host cells may be accomplished in any convenient manner, where two representative means of transformation are treatment with divalent cation transformation compositions and electrotransformation. In transformation through divalent cation treatment, the host cells are typically incubated with the one or more divalent cations, e.g. CaCl₂, which serves to make the host cell permeable to the vector DNA. See Cohen et al. (1972) Proc. Nat'l. Acad. Sci. USA 69:2110. Other agents with which the host cells may also be incubated include DMSO, reducing agents, hexaminecobalt and the like, where such agents serve to improve the efficiency of transformation. In electrotransformation (also known as transformation by electroporation) host cells are subject to an electrical pulse in the presence of the Vector in a manner sufficient for the vector to enter the host cells. See Dower et al. (1988) Nucleic Acids Research 16:6127.

A variety of host cells are suitable and may be used in the production of the VSHK-1 receptor polypeptides, where such host cells may be bacterial cells, yeast cells, or other cells, such as plant cells (see Depicker (1982) J. Mol. Appl. Gen. 1:561, where the host cell will generally be bacterial, e.g. E. coli, B. subtilis, where an E. coli strain is often the host cell of choice; or mammalian, e.g., COS, CHO, 3T3, and the like. E. coli strains that may be used include DH1, DH5, MM294, LE392, MC1061 and JM109.

Following transformation, bacterial host cells are screened for incorporation of the expression vector. Transformed colonies, e.g. host cells harboring the expression vector with the nucleic acid encoding the VSHK-1 receptor polypeptide are identified, and then grown up in large quantity. Where appropriate, agents that induce expression of the VSHK-1 receptor polypeptide are contacted with the host cell, e.g. isopropylthiogalactoside (IPTG).

Following colony growth, the expressed product will be harvested and purified for subsequent use. Typically, purification of the product involves disruption of the host cell, inactivation and removal of the native host proteins and precipitation of the nucleic acids. The product is separated from the other host cell constituents using one or more of a number of separation techniques known to those of skill in the art, e.g. centrifugation, dialysis, gel filtration chromatography, ion exchange chromatography, and the like. See Guide to Protein Purification (Murray P. Deutscher ed., Harcourt Brace & Co.)(1990). Using these protein purification techniques, isolated product may be prepared, where by isolated is meant a composition that is at least about 95% by weight peptidic product, usually at least about 98% by weight peptidic product and more usually at least about 99% by weight product, when the composition is dehydrated, e.g. lyophilized.

The subject nucleic acid molecules are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence.

Other vectors are suitable for expression in cells in culture. These vectors will generally include regulatory sequences (“control sequences” or “control regions”) which are necessary to effect the expression of a VSHK-1 polynucleotide to which they are operably linked. Still other vectors are suitable for transfer and expression in cells in a whole organism or person.

The VSHK-1 polynucleotides and receptor polypeptides of the present invention can be introduced into a cell by a gene delivery vehicle. Generally, gene delivery vehicles can encode either polypeptides or polynucleotides, such as antisense or ribozymes. The gene delivery vehicle may be of viral or non-viral origin (see generally, Jolly (1994) Cancer Gene Therapy 1:51-64; Kimura (1994) Human Gene Therapy 5:845-852; Connelly (1995) Human Gene Therapy 1:185-193; and Kaplitt (1994) Nature Genetics 6:148-153). Gene therapy vehicles for delivery of constructs including a coding sequence of a polynucleotide of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 415 731; WO 90/07936; WO 94/03622; WO 93/25698; WO 93/25234; U.S. Pat. No. 5, 219,740; WO 93/11230; WO 93/10218; Vile and Hart, Cancer Res. (1993) 53:3860-3864; Vile and Hart, Cancer Res. (1993) 53:962-967; Ram et al., Cancer Res. (1993) 53:83-88; Takamiya et al., J. Neurosci. Res. (1992) 33:493-503; Baba et al., J. Neurosurg. (1993) 79:729-735; U.S. Pat. No. 4,777,127; GB Patent No. 2,200,651; and EP 345 242.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. (1989) 63:3822-3828; Mendelson et al., Virol. (1988) 166:154-165; and Flotte et al., PNAS (1993) 90:10613-10617.

Also of interest are adenoviral vectors, e.g., those described by Berkner, Biotechniques (1988) 6:616-627; Rosenfeld et al.(1991) Science 252:431-434; WO 93/19191; Kolls et al. (1994) Proc. Natl. Acad. Sci. USA 91:215-219; Kass-Eisler et al. (1993) Proc. Natl. Acad. Sci. USA 90:11498-11502; WO 94/12649, WO 93/03769; WO 93/19191; WO 94/28938; WO 95/11984 and WO 95/00655.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell Biol. 14:2411-2418, and in Woffendin (1994) Proc. Natl. Acad. Sci. 91:1581-1585.

Naked DNA may also1be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 524 968.

Further non-viral delivery suitable for use includes mechanical delivery systems such as the approach described in Woffendin et al.(1994) Proc. Natl. Acad. Sci. USA 91:11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/1-1033.

Host Cells of the Invention

The present invention further provides host cells, which may be isolated host cells, comprising VSHK-1 polynucleotides of the invention. Suitable host cells include prokaryotes such as E. coli, B. subtilis, eukaryotes, including insect cells in combination with baculovirus vectors, yeast cells, such as Saccharomyces cerevisiae, or cells of a higher organism such as vertebrates, including amphibians (e.g., Xenopus laevis oocytes), and mammals, particularly mammals, e.g. COS cells, CHO cells, 293 cells, 3T3 cells, and the like, may be used as the expression host cells. Host cells can be used for the purposes of propagating a VSHK-1 polynucleotide, for production of a VSHK-1 receptor polypeptide, or in cell-based methods for identifying agents which modulate a level of VSHK-1 -mRNA and/or protein and/or enzyme activity in a cell.

The subject nucleic acids can be used to generate transgenic animals or site specific gene modifications in cell lines. The modified cells or animals are useful in the study of VSHK-1 function and regulation. For example, a series of small deletions or substitutions may be made in the VSHK-1 gene to determine the role of different coding regions in signal transduction, etc.

DNA constructs for homologous recombination will comprise at least a portion of the VSHK-1 gene with the desired genetic modification, and will include regions of homology to the target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line may be employed, or ES cells may be obtained freshly from a host, e.g. mouse, rat, guinea pig, etc. Such cells are grown on an appropriate fibroblast-feeder layer or grown in the presence of leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination. Those colonies that show homologous recombination may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. The chimeric animals are screened for the presence of the VSHK-1 gene and males and females having the modification are mated to produce homozygous progeny. The transgenic animals may be any non-human mammal, such as laboratory animals, domestic animals, etc. The transgenic animals may be used to determine the effect of a candidate drug on controlling unwanted cell proliferation, e.g., a reduction in cancer cell growth, in an in vivo environment.

Antibodies

Antibodies against VSHK-1 receptor polypeptides are useful for affinity chromatography, immunofluorescent assays, and detecting VSHK-1 receptor polypeptides. Further, antibodies can be useful to treat a VSHK-1 receptor mediated disorder. Antibodies which recognize the extracellular portions of native VSHK-1 receptors are of particular interest.

As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a VSHK-1 receptor polypeptide.

Antibodies to the proteins of the invention, both polyclonal and monoclonal, may be prepared by conventional methods. Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

If desired, the antibodies (whether polyclonal or monoclonal) may be labeled using conventional techniques. Suitable labels include fluorophores, chromophores, radioactive atoms (particularly ³²P and 125I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Compositions of the Invention

The present invention further provides compositions comprising the polypeptides, polynucleotides, recombinant vectors, host cells, and antibodies of the invention. These compositions may include a buffer, which is selected according to the desired use of the polypeptide, polynucleotide, recombinant vector, host cell, or antibody, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, Remington: The Science and Practice of Pharmacy, 19th Ed. (1995) Mack Publishing Co.

Methods using the Polypeptides and polynucleotides of the Invention

The present invention provides a variety of detection methods, which methods are useful in diagnostic assays. Also provided are assays for detecting VSHK-1 receptor ligands. Also provided are a variety of screening assays, which assays are useful for identifying agents which affect VSHK-1 receptor signal transduction or other activity, and/or VSHK-1 mRNA and/or VSHK-1 receptor polypeptide levels.

Detection Methods

Detection methods of the present invention include methods for detecting VSHK-1 receptor polypeptide in a biological sample, methods for detecting VSHK-1 mRNA in a biological sample, and methods for detecting VSHK-1 receptor signal transduction activity in a biological sample.

The detection methods can be provided as part of a kit. Thus, the invention further provides kits for detecting the presence and/or a level of VSHK-1 receptor polypeptide or VSHK-1 polynucleotides in a biological sample. Procedures using these kits can be performed by clinical laboratories, experimental laboratories, medical practioners, or private individuals. The kits of the invention for detecting a VSHK-1 receptor polypeptide comprise a moiety that specifically binds VSHK-1 receptor, including, but not limited to, a VSHK-1-specific antibody, and a VSHK-1 receptor ligand. The kits of the invention for detecting a VSHK-1 polynucleotide comprise a moiety that specifically hybridizes to a VSHK-1 polynucleotide. The kit may optionally provide additional components that are useful in the procedure, including, but not limited to, buffers, developing reagents, labels, reacting surfaces, means for detections, control samples, standards, instructions, and interpretive information.

Methods of Detecting a VSHK-1 Receptor Polypeptide in a Biological Sample

Immunoassays and ligand binding assays can be utilized to determine if a host cell is expressing the desired VSHK-1 receptor polypeptide.

For example, an immunofluorescence assay can be easily performed on host cells without separating the VSHK-1 receptor polypeptides from the cell membrane. The host cells are first fixed onto a solid support, such as a microscope slide or microtiter well. This fixing step can permeabilize the cell membrane. The permeablization of the cell membrane permits the antibodies to bind to the intracellular portions of native receptors, such as the second cytoplasmic loop of the VSHK-1 receptor polypeptides.

Next, the fixed host cells are exposed to an anti-VSHK-1 receptor polypeptide antibody. Preferably, to increase the sensitivity of the assay, the fixed cells are exposed to a second antibody, which is labeled and binds to the anti-VSHK-1 receptor polypeptide antibody. Typically, the secondary antibody is labeled with an fluorescent marker. The host cells which express the VSHK-1 receptor polypeptides will be fluorescently labelled and easily visualized under the microscope. See, for example, Hashido et al. (1992) Biochem. Biophys. Res. Comm. 187:1241-1248.

Also, the VSHK-1 receptor polypeptides do not need to be separated from the cell membrane for ligand binding assay. The host cells may be fixed to a solid support, such as a microtiter plate. Alternatively, a crude membrane fraction can be separated from lysed host cells by centrifugation (See Adachi et al. (1992) FEBS Lett. 311:179-183). The fixed host cells or the crude membrane fraction is exposed to labeled ligand, endogenous or non-natural. Typically, the ligand is labeled with radioactive atoms. The host cells which express the desired VSHK-1 receptor polypeptide can bind with the labeled ligand which can be easily detected.

Alternatively, whole cells, expressing VSHK-1 receptor polypeptides, can be cultured in a microtiter plate, for example, and used for ligand binding assay. See Sakamoto et al. (1991) Biochem. Biophys. Res. Comm. 178:656-663 for a description of such an assay.

Other assays can be used to determine if a host cell is expressing a VSHK-1 receptor polypeptide, such as signal transduction assays and immunoassays. These assays are described below.

The present invention further provides methods for detecting the presence of and/or measuring a level of a VSHK-1 receptor polypeptide in a biological sample, using a VSHK-1 receptor-specific antibody. The methods generally comprise:
a) contacting the sample with an antibody specific for a VSHK-1 receptor polypeptide; and
b) detecting binding between the antibody and molecules of the sample.

Detection of specific binding of the VSHK-1 receptor-specific antibody, when compared to a suitable control, is an indication that VSHK-1 receptor polypeptides are present in the sample. Suitable controls include a sample known not to contain a VSHK-1 receptor polypeptide; and a sample contacted with an antibody not specific for VSHK-1 receptor, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the VSHK-1 receptor-specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. The antibody may be attached (coupled) to an insoluble support, such as a polystyrene plate or a bead. Indirect labels include second antibodies specific for VSHK-1 receptor-specific antibodies, wherein the second antibody is labeled as described above; and members of specific binding pairs, e.g., biotin-avidin, and the like. The biological sample may be brought into contact with an immobilized on a solid support or carrier, such as nitrocellulose, that is capable of immobilizing cells, cell particles, or soluble proteins. The support may then be washed with suitable buffers, followed by contacting with a detectably-labeled VSHK-1 receptor-specific antibody. Detection methods are known in the art and will be chosen as appropriate to the signal emitted by the detectable label. Detection is generally accomplished in comparison to suitable controls, and to appropriate standards.

Methods of Detecting a VSHK-1 mRNA in a Biological Sample

The present invention further provides methods for detecting the presence of VSHK-1 mRNA in a biological sample. The methods can be used, for example, to assess whether a test compound affects VSHK-1 gene expression, directly or indirectly.

The methods generally comprise:
a) contacting the sample with a VSHK-1 polynucleotide of the invention under conditions which allow hybridization; and
b) detecting hybridization, if any.

Detection of hybridization, when compared to a suitable control, is an indication of the presence in the sample of a VSHK-1 polynucleotide. Appropriate controls include, for example, a sample which is known not to contain VSHK-1 mRNA, and use of a labelled polynucleotide of the same “sense” as a VSHK-1 mRNA. Conditions which allow hybridization are known in the art, and have been described in more detail above. Detection can be accomplished by any known method, including, but not limited to, in situ hybridization, PCR, RT-PCR, and “Northern” or RNA blotting, or combinations of such techniques, using a suitably labelled VSHK-1 polynucleotide. A variety of labels and labelling methods for polynucleotides are known in the art and can be used in the assay methods of the invention. Specific hybridization can be determined by comparison to appropriate controls.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp.14.2-14.33. A detectable label may be included in the amplification reaction. Suitable labels include fluorochromes, e.g. fluorescein isothiocyanate (FITC), rhodamine, Texas Red, phycoerythlrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMPA) radioactive labels, e.g. ³²P, ³⁵S, ³H; etc. The label may be a two stage system, where the amplified DNA is conjugated to biotin, haptens, etc. having a high affnity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Screening Assays

The present invention provides screening methods for identifying agents which modulate VSHK-1 receptor signal transduction activity, methods for identifying agents which are VSHK-1 receptor ligands, methods for identifying agents which modulate a level of VSHK-1 receptor polypeptide in a cell, and methods for identifying agents which modulate a level of VSHK-1 mRNA in a cell.

The terms “agent”, “substance” and “compound” are used interchangeably herein. Candidate agents encompass numerous chemical classes, typically synthetic, semi-synthetic, or naturally-occurring inorganic or organic molecules. Candidate agents may be small organic compounds having a molecular weight of more than 50 and less than about 2,500 daltons. Candidate agents may be peptides, or peptoids. Candidate agents may comprise functional groups necessary for structural interaction with proteins, particularly hydrogen bonding, and typically include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

Methods of Detecting VSHK-1 Receptor Ligands, Agonists, and Antagonists

VSHK-1 receptor polypeptides can also be used to screen libraries of compounds, such as peptide libraries, to identify receptor binding moieties. Receptor binding moieties includes ligands, which may be receptor agonists or antagonists. Accordingly, the screening methods of the invention include methods for identifying VSHK-1 receptor polypeptide ligands, and methods for identifying agents that effect or modulate a VSHK-1 receptor signal transduction activity. As used herein, the term “modulate” encompasses “increase” and “decrease”. Assays are conducted in vitro, and can be cell-based or cell-free.

Methods-to identify VSHK-1 receptor ligands generally comprise a) contacting a substance to be tested with a sample containing a VSHK-1 receptor polypeptide; and b) assaying a signal transduction activity of the VSHK-1 receptor polypeptide in the presence of the substance. Signal transduction in the presence of the substance is an indication that the substance is a VSHK-1 receptor ligand.

Methods to identify substances which modulate a signal transduction activity of a VSHK-1 receptor polypeptide generally comprise: a) contacting a substance to be tested with a sample containing a VSHK-1 receptor polypeptide; and b) assaying a signal transduction activity of the VSHK-1 receptor polypeptide in the presence of the substance. An increase or a decrease in signal transduction activity in comparison to VSHK-1 receptor signal transduction activity in a suitable control (e.g., a sample comprising a VSHK-1 receptor polypeptide in the absence of the substance being tested) is an indication that the substance modulates a signal transduction activity of the VSHK-1 receptor polypeptide. An increase in signal transduction activity indicates that the substance is a VSHK-1 receptor agonist. An agonist generally promotes signal transduction nearly as well, as well, or better, than a known ligand. A decrease in signal transduction activity indicates that the substance is a VSHK-1 receptor antagonist. To identify a receptor antagonist, the assay results are generally compared to a signal transduction level mediated by a known ligand.

Signal transduction activity can be assayed by measuring a level of one or more of: intracellular Ca²⁺, intracellular IP3, and intracellular DAG, as described in detail below. Signal transduction activity may be measured in vitro in intact eukaryotic cells, or, alternatively, in cell-free membrane preparations. The cells used may be cells which express endogenous VSHK-1 receptor polypeptide in the cell membrane. Alternatively, the cell may be transduced with a recombinant construct comprising a nucleotide sequence encoding a VSHK-1 receptor polypeptide, as long as other components necessary for signal transduction activity of VSHK-1 receptor are present in the cell. In these situations, the VSHK-1 receptor polypeptide-encoding nucleotide sequence may be operably linked to a heterologous promoter, e.g., an inducible promoter, a constitutive promoter, and the like, depending on the assay.

In some embodiments, the candidate agent is a peptide. Various methods have been described for identifying a receptor-binding peptide, any of which may be suitable for use herein. Following are two non-limiting examples of suitable methods.

A “library” of peptides may be synthesized following the methods disclosed in U.S. Pat. No. 5,010,175. Briefly, one prepares a mixture of peptides, which is then screened to determine the peptides exhibiting the desired signal transduction and receptor binding activity. In the method disclosed in U.S. Pat. No. 5,010,175, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g. hexamers) is formed. Note that one need not include all amino acids in each step: one may include only one or two-amino acids in some steps (e.g., where it is known that a particular amino acid is essential in a given position), thus reducing the complexity of the mixture. After the synthesis of the peptide library is completed; the mixture of peptides is screened for binding to the selected VSHK-1 receptor polypeptide. The peptides are then tested for their ability to inhibit or enhance VSHK-1 receptor signal transduction activity. Peptides exhibiting the desired activity are then isolated and sequenced.

Another method for identifying receptor-binding peptides is provided in PCT W091/17823. This method is similar to the method described in U.S. Pat. No. 5,010,175. However, instead of reacting the synthesis resin with a mixture of activated amino acids, the resin is divided into twenty equal portions (or into a number of portions corresponding to the number of different amino acids to be added in that step), and each amino acid is coupled individually to its portion of resin. The resin portions are then combined, mixed, and again divided into a number of equal portions for reaction with the second amino acid. In this manner, each reaction may be easily driven to completion. Additionally, one may maintain separate “sub-pools” by treating portions in parallel, rather than combining all resins at each step. This simplifies the process of determining which peptides are responsible for any observed receptor binding or signal transduction activity.

In such cases, the subpools containing, e.g., 1-2,000 candidates each are exposed to the desired VSHK-1 receptor polypeptide. Each subpool that produces a positive result is then resynthesized as a group of smaller subpools (sub-subpools) containing, e.g., 20-100 candidates, and reassayed. Positive sub-subpools may be resynthesized as individual compounds, and assayed finally to determine the peptides, which exhibit a high binding constant. Then, these peptides can be tested for their ability to inhibit or enhance the VSHK-1 receptor signal transduction activity. The methods described in '17823 and U.S. Pat. No. 5,194,392 (herein incorporated by reference) enable the preparation of such pools and subpools by automated techniques in parallel, such that all synthesis and resynthesis may be performed in a matter of days.

Compounds which act as VSHK-1 receptor binding ligands, agonists, and antagonists are screened using any known method. The methods described herein are presently preferred. The assay conditions ideally should resemble the conditions under which the VSHK-1 receptor signal transduction is exhibited in vivo, i.e., under physiologic pH, temperature, ionic strength, etc. Suitable agonists or antagonists will exhibit strong inhibition or enhancement of the VSHK-1 receptor signal transduction activity at concentrations which do not raise toxic side effects on the cell or in the subject. Agonists or antagonists which compete for binding to the VSHK-1 receptor ligand binding site may require concentrations equal to or greater than the VSHK-1 receptor concentration, while inhibitors capable of binding irreversibly to the VSHK-1 receptor may be added in concentrations on the order of the VSHK-1 receptor concentration.

Signal Transduction Assays

Signal transduction assays and other biological assays are useful for identifying agonists and antagonists, including mutants, fragments and fusions of the native VSHK-1. Signal transduction can be measured by Ca+release, inositol triphosphate concentration, and/or cAMP concentration. Signal transduction protocols are known to those skilled in the art, and have been amply described in various publications, including, for example, Signal Transduction Protocols: Methods in Molecular Biology, Vol. 41, Kendall and Hill, Eds, Humana Press, 1995; and Signalling by Inositides: A Practical Approach, Shears, Ed., IRL Press, 1997.

Most cellular Ca²⁺ ions are sequestered in the mitochondria, endoplasmic reticulum, and other cytoplasmic vesicles, but binding of ligand to VSHK-1 will trigger the increase of free Ca²⁺ ions in the cytoplasm. With fluorescent dyes, such as fura-2, the concentration of free Ca²⁺ can be monitored. The ester of fura-2 is added to the media of the host cells expressing VSHK-1 receptor polypeptides. The ester of fura-2 is lipophilic and diffuses across the membrane. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to the free Ca²⁺ ions, which are released after binding of a ligand to a VSHK-1 receptor polypeptide. The fluorescence can be measured without lysing the cells at an excitation spectrum of 340 nm or 380 nm-and at fluorescence spectrum of 500 nm. See Sakurai et al., EP 480 381 and Adachi et al., FEBS Lett 311(2): 179-183 (1992) for examples of assays measuring free intracellular Ca²⁺ concentrations.

The rise of free cytosolic Ca²⁺ concentrations is preceded by the hydrolysis of phosphatidylinositol 4,5-bisphosphate. Hydrolysis of this phospholipid by the plasma-membrane enzyme phospholipase C yields 1,2-diacylglycerol (DAG), which remains in the membrane, and the water-soluble inositol 1,4,5-trisphosphate (IP₃). Binding of endogenous ligand or agonists will increase the concentration of DAG and IP₃. Thus, signal transduction activity can be measured by monitoring the concentration of these hydrolysis products.

To measure the IP₃ concentrations, radioactively labelled ³H-inositol is added to the media of host cells expressing VSHK-1 receptor polypeptides. The ³H-inositol taken up by the cells and after stimulation of the cells with endogenous ligand or agonist, the resulting inositol triphosphate is separated from the mono and di-phosphate forms and measured. See Sakurai et al., EP 480 381. Alternatively, Amersham provides an inosital 1,4,5-trisphosphate assay system. With this system Amersham provides tritylated inosital 1,4,5-trisphosphate and a receptor capable of distinguishing the radioactive inositol from other inositol phosphates. With these reagents an effective and accurate competition assay can be performed to determine the inositol triphosphate levels.

Methods of Detecting Agents which Modulate a Level of VSHK-1 mRNA and/or VSHK-1 Receptor Polypeptide

A wide variety of cell-based assays may be used for identifying agents which modulate levels of VSHK-1 mRNA, using, for example, a mammalian cell transformed with a construct comprising a VSHK-1 receptor-encoding cDNA such that the cDNA is overexpressed, or, alternatively, a construct comprising a VSHK-1 promoter operably linked to a reporter gene.

Accordingly, the present invention provides a method for identifying an agent, particularly a biologically active agent, that modulates a level of VSHK-1 expression in a cell, the method comprising: combining a candidate agent to be tested with a cell comprising a nucleic acid which encodes a VSHK-1 receptor polypeptide; and determining the effect of said agent on VSHK-1 expression. “Modulation” of VSHK-1 expression levels includes increasing the level and decreasing the level of VSHK-1 mRNA and/or VSHK-1 receptor polypeptide encoded by the VSHK-1 polynucleotide when compared to a control lacking the agent being tested. An increase or decrease of about 1.25-fold, usually at least about 1.5-fold, usually at least about 2-fold, usually-at least about 5-fold, usually at least about 10-fold or more, in the level (i.e., an amount) of VSHK-1 mRNA and/or polypeptide following contacting the cell with a candidate agent being tested, compared to a control to which no agent is added, is an indication that the agent modulates VSHK-1 expression.

An agent being tested for its effect on VSHK-1 expression is assessed for any cytotoxic activity it may exhibit toward the cell used in the assay, using well-known assays, such as trypan blue dye exclusion, an MTT ([3-(4,5-dimethylthiazol-2-yl)-2,5-diphenyl-2 H-tetrazolium bromide]) assay, and the like. Agents that do not exhibit cytotoxic activity are considered candidate agents.

The cells used in the assay are usually mammalian cells, including, but not limited to, rodent cells and human cells. The cells may be primary cell cultures or may be immortalized cell lines.

VSHK-1 mRNA and/or polypeptide whose levels are being measured can be encoded by an endogenous VSHK-1 polynucleotide, or the VSHK-1 polynucleotide can be one that is comprised within a recombinant vector and introduced into the cell, i.e., the VSHK-1 mRNA and/or polypeptide can be encoded by an exogenous VSHK-1 polynucleotide. For example, a recombinant vector may comprise an isolated VSHK-1 transcriptional regulatory sequence, such as a promoter sequence, operably linked to a reporter gene (e.g,. β-galactosidase, CAT, luciferase, or other gene that can be easily assayed for expression). In these embodiments, the method for identifying an agent that modulates a level of VSHK-1 expression in a cell, comprises: combining a candidate agent to be tested with a cell comprising a nucleic acid which comprises a VSHK-1 gene transcriptional regulatory element operably linked to a reporter gene; and determining the effect of said-agent on reporter gene expression. A recombinant vector may comprise an isolated VSHK-1 transcriptional regulatory sequence, such as a promoter sequence, operably linked to sequences coding for a VSHK-1 receptor polypeptide; or the transcriptional control sequences can be operably linked to coding sequences for a VSHK-1 fusion protein comprising VSHK-1 receptor polypeptide fused to a polypeptide which facilitates detection. In these embodiments, the method comprises combining a candidate agent to be tested with a cell comprising a nucleic acid which comprises a VSHK-1 gene transcriptional regulatory element operably linked to a VSHK-1 receptor polypeptide-coding sequence; and determining the effect of said agent on VSHK-1 expression, which determination can be carried out by measuring an amount of VSHK-1 mRNA, VSHK-1 receptor polypeptide, or VSHK-1 fusion polypeptide produced by the cell.

Cell-based assays generally comprise the steps of contacting the cell with an agent to be tested, forming a test sample, and, after a suitable time, assessing the effect of the agent on VSHK-1 expression. A control sample comprises the same cell without the candidate agent added. VSHK-1 expression levels are measured in both the test sample and the control sample. A comparison is made between VSHK-1 expression level in the test sample and the control sample. VSHK-1 expression can be assessed using conventional assays. For example, when a mammalian cell line is transformed with a construct that results in expression of VSHK-1, VSHK-1 mRNA levels can be detected and measured, as described above, or VSHK-1 receptor polypeptide levels can be detected and measured, as described above. A suitable period of time for contacting the agent with the cell can be determined empirically, and is generally a time sufficient to allow entry of the agent into the cell and to allow the agent to have a measurable effect on VSHK-1 mRNA and/or polypeptide levels. Generally, a suitable time is between 10 minutes and 24 hours, more typically about 1-8 hours: Methods of measuring VSHK-1 mRNA levels are known in the art, several of which have been described above, and any of these methods can be used in the methods of the present invention to identify an agent which modulates VSHK-1 mRNA level in a cell, including, but not limited to, a PCR, such as a PCR employing detectably labeled oligonucleotide primers, and any of a variety of hybridization assays. Similarly, VSHK-1 receptor polypeptide levels can be measured using any standard method, several of which have been described herein, including, but not limited to, an immunoassay such as ELISA, for example an ELISA employing a detectably labeled antibody specific for a VSHK-1 receptor polypeptide.

Compositions Comprising Identified Substances

The present invention further provides substances identified by any of the above-described screening methods. The substances may be provided in a composition comprising the substance(s). These compositions may include a buffer, which is selected according to the desired use of the substance, appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19th Ed. (1995) Mack Publishing Co.

Treatment Methods

Treatment of VSHK-1 Receptor Mediated Disorders

Methods of treatment or amelioration include administering compositions of polynucleotides, polypeptides, antibodies, or combinations thereof and can be used
to inhibit translation and/or transcription;
to inhibit biological activity;
as a vaccine antigen; and
as an immune system inducer.
   Such compositions can be administered systemically or locally to the desired site. For example, modulation of genes or gene expression products that are mis-regulated can be used to treat VSHK-1 receptor mediated disorders and/or the accompanying physical and biological manifestations.

Therapeutic compositions may comprise one or more of the following: (1) ribozymes and/or antisense molecules that reduce the level of VSHK-1 mRNA in a cell; (2) a construct comprising nucleotide sequences encoding a VSHK-1 receptor polypeptide; (3) an antibody of the invention; (4) a VSHK-1 receptor polypeptide of the invention; (5) a VSHK-1 ligand; (6) a VSHK-1 receptor agonist; (7) a VSHK-1 antagonist.

The methods generally comprise contacting a eukaryotic cell with a substance which, after entering the cell, inhibits and/or modulates a signal transduction activity of a VSHK-1 receptor polypeptide, and/or which modulates a level of VSHK-1 mRNA and/or VSHK-1 receptor polypeptide in the eukaryotic cell. Generally, the cell is contacted with a composition comprising an effective amount of the substance.

An effective amount of a substance which modulates a signal transduction activity of a VSHK-1 receptor polypeptide is an amount that increases or decreases signal transduction activity of a VSHK-1 receptor polypeptide by at least about 10%, more preferably at least about 15%, more preferably at least about 25%, more preferably at least about 50% or more, when compared to the signal transduction activity of the VSHK-1 receptor polypeptide in the absence of the substance. An effective amount of a substance which modulates a level of VSHK-1 mRNA in a cell is an amount that increases or reduces VSHK-1 mRNA level by at least about 10%, more preferably at least about 15%, more preferably at least about 25%, more preferably at least about 50% or more, when compared to the level of VSHK-1 mRNA in the absence of the substance. An effective amount of a substance which modulates a level of VSHK-1 receptor polypeptide in a cell is an amount that increases or reduces a VSHK-1 receptor polypeptide level by at least about 10%, more preferably at least about 15%, more preferably at least about 25%, more preferably at least about 50% or more, when compared to the level of VSHK-1 receptor polypeptide in the absence of the substance.

Cells which are targets for the methods of the present invention are those which (1) express VSHK-1 mRNA and/or polypeptide at lower than normal levels; (2) express a VSHK-1 receptor polypeptide with abnormal function; or (3) normally express VSHK-1 receptor polypeptide, such as heart cells.

Pharmaceutical Compositions and Therapeutic Uses

Pharmaceutical compositions can comprise polypeptides; antibodies; VSHK-1 ligands, antagonists, or agonists; and/or polynucleotides of the invention. The pharmaceutical compositions will comprise a therapeutically effective amount of either polypeptides, antibodies, or polynucleotides of the invention.

The term “therapeutically effective amount” as used herein refers to an amount of a therapeutic agent to treat, ameliorate, or prevent a desired disease or condition, or to exhibit a detectable therapeutic or preventive effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the polynucleotide, polypeptide or antibody compositions in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington 's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

The therapeutic agents may be administered in a variety of ways, orally, topically, parenterally e.g. subcutaneously, intraperitoneally, by viral infection, intravascularly, etc. Inhalation treatments may also be of interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Once formulated, the polynucleotide compositions of the invention can be (1) administered directly to the subject; (2) delivered ex vivo, to cells derived from the subject; or (3) delivered in vitro for expression of recombinant proteins.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

The present invention will now be illustrated by reference to the following examples which set forth particularly advantageous embodiments. However, it should be noted that these embodiments are illustrative and are not to be construed as restricting the invention in any way.

### EXAMPLES

### Example 1

cDNA Cloning and Characterization of VSHK-1

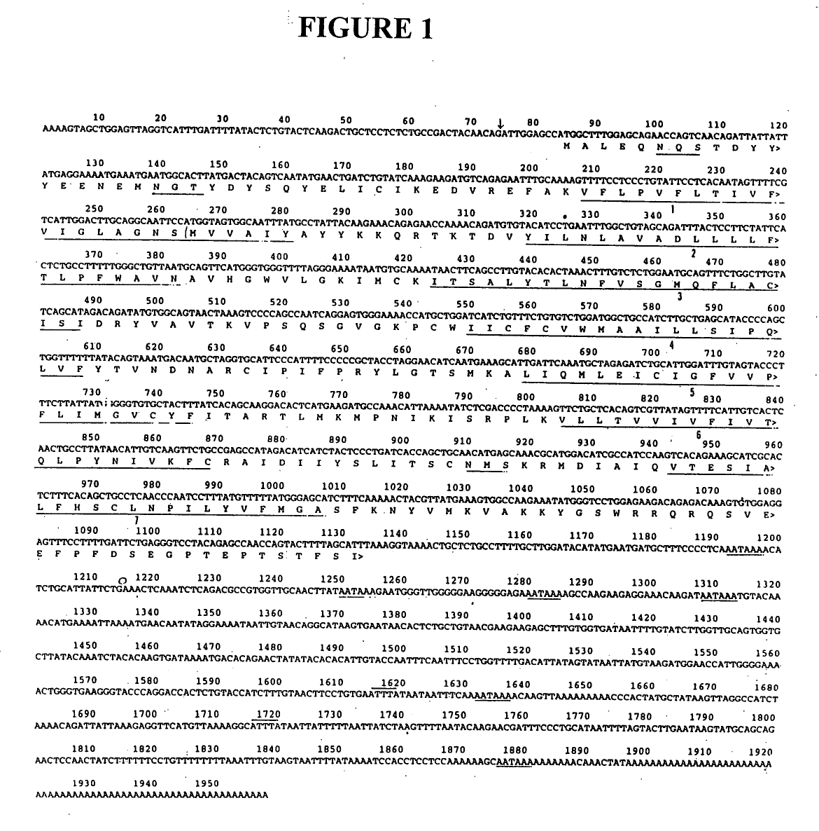

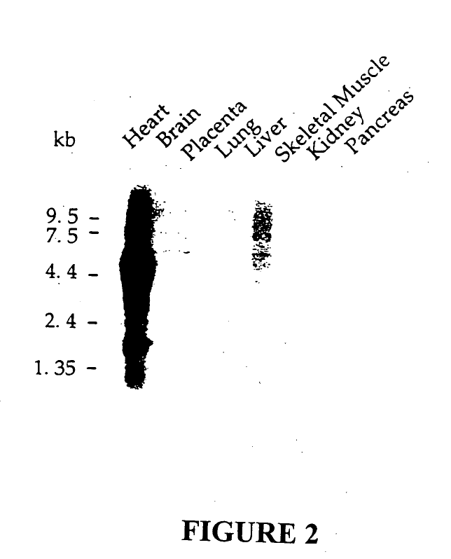

VSHK-1 cDNA was cloned and sequenced. As shown in FIG. 1 the cDNA sequence (SEQ ID NO:1) has two potential Met start codons, the first of which has a consensus Kozak sequence. Full-length VSHK-1 cDNA encodes a polypeptide of 350 amino acids (SEQ ID NO:2), with several conserved motifs typical of seven transmembrane receptors. In FIG. 2, the putative transmembrane domains are underlined and labeled 1 through 7. The amino acid sequence has three potential N-glycosylation sites, two in the amino terminal portion (underlined “NQS” and “NGT” in FIG. 1 and one in the third extracellular loop (underlined “NMS” in FIG. 1. EST H67224 (GenBank Accession No. H67224) provides a 328-nucleotide sequence which is identical (except for the “n”, or unidentified, nucleotide in EST H67224) to nucleotides 324 through 651 of SEQ ID NO:1.

As shown in FIG. 2, VSHK-1 mRNA is found predominantly in heart tissue. Faint or undetectable hybridization was detected with RNA from brain, placenta, lung, liver, skeletal muscle, kidney, and pancreas. In heart, three RNA species were identified: a 1.3 kb; a 2.0 kb; and a 5.0 kb species. The cDNA depicted in FIG. 1 corresponds to the 2.0 kb form. Use of an alternative polyadenylation site could account for the 1.3 form.

Genomic analysis revealed an intron at nucleotide 74, with splice donor and acceptor sites being ACTACCAACAGgttggtacttta (SEQ ID NO:3) and ctttgccatctagAGTGGAGCC (SEQ ID NO:4), respectively. The 3.0 kb intron is transcribed, and could account for the 5.0 kb mRNA species.

The amino acid sequence of VSHK-1 was compared to known amino acid sequences available in GenBank. Using the Clustal W program with default parameters, sequences sharing amino acid identity with VSHK-1 were identified as CCR6, CCR7, and CXCR2. CCR6 and CCR7 share 32% and 37% amino acid sequence identity, respectively, with the sequence set forth in SEQ ID NO:2. The alignment is shown in FIG. 3. The highest amino acid sequence identity is found in transmembrane domain 2.

The VSHK-1 coding region was cloned into the mammalian expression vector pcDNA3 (Invitrogen). The construct, designated pHA-VSHK1, also includes a hemagluttinin (HA) epitope tag in-frame with VSHK-1 coding sequences. The insert was transcribed in vitro and translated in the presence and absence of canine pancreatic microsomal membranes. A major polypeptide of approximately 45 kDa was observed, which was modified in the presence-of the membrane to a higher molecular weight form. The presence of the additional HA tag at the N-terminus increases the size of-the polypeptide, confirming that the signal sequence is not processed.

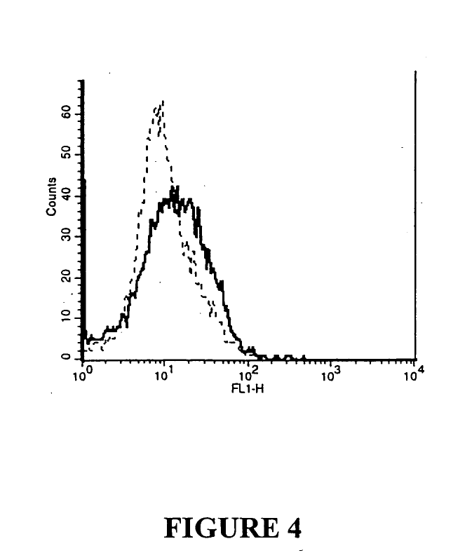

HEK-293 cells were transiently transfected with either pHA-VSHK-1 or pcDNA-3 (vector control), using TransIt-LT1. Shymala and Khoja (1998) Biochem. 37:15918-15924. Six hours post-transfection, the media was changed to DMEM containing 10% serum. 48 hours later, cells were released from the plates with Ca²⁺ and Mg²⁺-free phosphate buffered saline containing-5 mM EDTA. Intact cells were treated with mouse monoclonal 12CA5 anti-HA antibody (Boehringer), followed by incubation with FITC-conjugated anti-mouse antibody. The cell suspension was subjected to FACS (fluorescence-activated cell sorting) analysis. The results are shown in FIG. 4. Vector control (dashed lines) shows background binding, while pHA-VSHK-1 expressing cells (solid lines) showed expression of the epitope-tagged protein.

### Example 2

Identification of Receptor Binding Agonists and Antagonists

VSHK-1 peptide ligands, agonists, and antagonists can be identified using the following methods. Construction of a phage library encoding random peptides is described in Devlin, WO91/18980. Such a construction consists of
(1) Producing Oligonucleotides Encoding Random Peptides;
(2) Creating a Shuttle Vector, Plasmid M13LP67, for Recombination with the Wild Type Phage; and
(3) Production of Phage Encoding Random Peptides by Recombination.
   Once the phage library is constructed, the library is screened using a VSHK-1 receptor polypeptide. From the phage library, peptides with the desired binding properties can be assayed for their receptor binding agonist or antagonist properties.
   I. Producing Oligonucleotides Encoding Random Peptides

An oligonucleotide having the following structure was synthesized, and purified using methods known in the art, as described in Devlin, WO91/18980:

(SEQ ID NO: 5)

5′
CTTTCTATTCTCACTCCGCTGAA(NNS)₁₅CCGCCTCCACCTCCACC 3′;
and

(SEQ ID NO: 6)

5′
GGC CGG TGG AGG TGG AGG CGG (iii)₁₅ TTC AGC GGA GTG
AGA ATA GAA AGG - TAC 3′.

During the synthesis of (NNS)₁₅, a mixture consisting of equal amounts of the deoxy-nucleotides A, C and T, and about-30% more G was used for N, and an equal mixture of C and G for S. Deoxyinosine (i) was used because of its capacity to base pair with each of the four bases (A, G, C, and T) (J. F. Reidhaar-Olson et al., Science, (1988) 24:53). Alternatively, other base analogs may be used as described by J. Habener et al., Proc Natl Acad Sci USA (1988) 85:1735.

Immediately preceding the nucleotide sequence that encodes the random peptide sequence is a nucleotide sequence that encodes alanine and glutamic acid residues. These amino acids were included because they correspond to the first two amino terminal residues of the wild type mature gene III protein of M13, and thus may facilitate producing the fusion protein produced as described below.

Immediately following the random peptide sequence is a nucleotide sequence that encodes 6 proline residues. Thus, the oligonucleotide encodes the following amino acid sequence:
H₂N-Ala-Glu-Xaa₁₅-Pro₆.

Xaa denotes amino acids encoded by the random DNA sequence. As described below, the oligonucleotides were cloned into a derivative of M13 to produce a mature fusion protein having the above amino acid sequence, and following the proline residues, the entire wild type mature gene III.

II. Construction the Shuttle Vector. Plasmid M13LP67. for Recombination with the Wild Type Phage

The plasmid M13LP67 was used to express the random peptide/gene III fusion protein construct. M13LP67 was derived from M13 mp19.

Briefly, M13mp19 was altered in two ways. The first alteration consisted of inserting the marker gene, β-lactamase, into the polylinker region of the virion. This consisted of obtaining the gene by PCR amplification from the plasmid pAc5. The oligonucleotide primers that were annealed to the pAc5 template have the following sequence:

| | |
|---|---|
| 5′ GCT GCC CGA GAG ATC TGT ATA TAT | (SEQ ID NO: 7) |
| GAG TAA ACT TGG 3′; | |
| and | |
| 5′ GCA GGC TCG GGA ATT CGG GAA ATG | (SEQ ID NO: 8) |
| TGC GCG GAA CCC 3′. | |

Amplified copies of the β-lactamase gene were digested with the restriction enzymes BglII and EcoRI, and the replicative form of the modified M13mp 19 was digested with BamHI and EcoRI. The desired fragments were purified by gel electrophoresis, ligated, and transformed into E. coli strain DH5 alpha (BRL). E. coli transformed with phage that carried the insert were selected on ampicillin plates. The phage so produced were termed JD32.

The plasmid form of the phage, pJD32 (M13mp19Amp^{r}), was mutagenized so that two restriction sites, EagI and KpnI, were introduced into gene III without altering the amino acids encoded in this region. The restriction sites were introduced using standard PCR in vitro mutagenesis techniques as described by M. Innis et al. in “PCR Protocols—A Guide to Methods and Applications” (1990), Academic Press, Inc.

The KpnI site was constructed by converting the sequence, TGTTCC, at position 1611 to GGTACC. The two oligonucleotides used to effect the mutagenesis have the following sequence:

| | | |
|---|---|---|
| LP159: | AAACTTCCTCATGAAAAAGTC; | (SEQ ID NO: 9) |
| and | | |
| LP162: | AGAATAGAAAGGTACCACTAAAGGA. | (SEQ ID NO: 10) |

To construct the EagI restriction site, the sequence at position 1631 of pJD32, CCGCTG, was changed to CGGCCG using the following two oligonucleotides:

| | |
|---|---|
| LP160: | |
| TTT AGT GGT ACC TTT CTA TTC TCA | (SEQ ID NO: 11) |
| CTC GGC CGA AAC TGT; | |
| and | |
| LP161: | |
| AAA GCG CAG TCT CTG AAT TTA CCG. | (SEQ ID NO: 12) |

More specifically, the PCR products obtained using the primers LP159, LP162 and LP160 and LP161 were digested with BspHI and KpnI, and KpnI and AlwNI, respectively. These were ligated with T4 ligase to M13mp19 previously cut with BspHI and AlwNI to yield M13mpLP66. This vector contains the desired EagI and KpnI restriction sites, but lacks the ampicillin resistance gene, β-lactamase. Thus, the vector M13mpLP67, which contains the EagI and KpnI restriction sites and β-lactamase was produced by removing the β-lactamase sequences from pJD32 by digesting the vector with XbaI and EcoRI. The β-lactamase gene was then inserted into the polylinker region of M13mpLP66 which was previously digested with XbaI and EcoRI. Subsequent ligation with T4 ligase produced M13mpLP67, which was used to generate the random peptide library. Schematics of the construction of M13mpLP67 are shown in Devlin et al., PCT WO91/18980.

Production of Phase Encoding Random Peptides

To produce phage having DNA sequences that encode random peptide sequences, M13LP67 was digested with EagI and KpnI, and ligated to the oligonucleotides. The ligation mixture consisted of digested M13LP67 DNA at 45 ng/μl, a 5-fold molar excess of oligonucleotides, 3.6 U/μl of T4 ligase (New England Biolabs), 25 mM Tris-HCl, pH 7.8, 10 mM MgCl₂, 2 mM DTT, 0.4 mM ATP, and 0.1 mg/ml BSA. Prior to being added to the ligation mixture, the individual oligonucleotides were combined and heated to 95° C. for 5 minutes, and subsequently cooled to room temperature in 15 μL aliquots. Next, the ligation mixture was incubated for 4 hours at room temperature and subsequently overnight at 15° C. This mixture was then electroporated into E. coli as described below.

M13LP67 DNA was electroporated into H249 cells prepared essentially as described by W. Dower et al.(1988) Nucl. Acids Res. 16:6127. H249 cells are a recA, sup°, F′ kan^{R} derivative of MM294. Briefly, 4×10⁹ H249 cells and 1 μg of M13LP67 DNA were combined in 85 μl of a low conductivity solution consisting of 1 mM HEPES. The cell/M13LP67DNA mixture was positioned in a chilled 0.56 mm gap electrode of a BTX electroporation device (BTX Corp.) and subjected to a 5 millisecond pulse of 560 volts.

Immediately following electroporation, the cells were removed from the electrode assembly, mixed with fresh H249 lawn cells, and plated at a density of about 2×10⁵ plaques per 400 cm² plate. The next day phage from each plate were eluted with 30 ml of fresh media, PEG precipitated, resuspended in 20% glycerol, and stored frozen at −70° C. About 2.8×10⁷ plaques were harvested and several hundred analyzed to determine the approximate number that harbor random peptide sequences.

Using the polymerase chain reaction to amplify DNA in the region that encodes the random peptide sequence, it was determined that about 50-90% of the phage contained a 69 base pair insert at the 5′ end of gene III. This confirmed the presence of the oligonucleotides that encode the random peptides sequences. The PCR reaction was conducted using standard techniques and with the following oligonucleotides:

| | |
|---|---|
| 5′ TCGAAAGCAAGCTGATAAACCG 3′; | (SEQ ID NO: 13) |
| and | |
| 5′ ACAGACAGCCCTCATAGTTAGCG 3′. | (SEQ ID NO: 14) |

The reaction was run for 40 cycles, after which the products were resolved by electrophoresis in a 2% agarose gel. Based on these results, it was calculated that phage from the 2.8×10⁷ plaques encode about 2×10⁷ different random amino acid sequences.

Panning for Receptor Binding Agonists and Antagonists

Peptides having an affinity for VSHK-1 receptor are identified as follows:
1.) 15 mer phage (2.5×10¹⁰) prepared as described above are selected by coincubation with 10⁶ Sf9 (Spodoptera frugiperda) cells expressing native VSHK-1 in a baculovirus expression vector on the second day after infection. The coincubation is at room temperature for 60 minutes in Grace's medium with 2% nonfat milk. Binding phage are eluted with 6M urea (pH 2.2), the pH neutralized by adding 2 M Tris-HCl, and assayed. The phage are amplified on solid agar plates as plaques, eluted with Tris-buffered saline, and precipitated with polyethylene glycol.
2.) The phage resulting from round 1 are reselected on CHO cells expressing the native VSHK-1 on second day 2 after plating the cells at a density of 7.1×10⁵, using 3.1×10¹¹ phage on in DMEM with 2% nonfat milk and 10 mM HEPES. The phage are bound, eluted, assayed, and amplified as described in round 1.
3.) The phage selected in round 2 are reselected on Sf9 cells expressing the native VSHK-1 receptor on day 2 post-infection as described for round 1 (2.8×10¹⁰ phage on 10⁶ Sf9 cells). Sample phage from the urea eluate are cloned, and their DNAs are isolated and sequenced.

Once the amino acid sequence of the putative agonists and antagonists is determined, synthetic oligopeptides can be produced and their signal transduction activity can be assayed by, for example, Amersham's inositol 1,4,5-trisphosphate assay system (Arlington Heights, Ill., U.S.A.).

CHO cells expressing the VSHK-1 receptor polypeptide are plated at a density of 1×10⁵ cells/well in a 12-well plate. The cells are cultured for 2 days, and then, the cells are washed twice with PBS containing 0.2% BSA. Next, the cells are incubated in the same medium for 30 minutes at 37° C. The medium on the cells is changed to PBS containing 0.2% BSA and 10 mM LiCl, and the cells are incubated for another 30 minutes at 37° C.

Signal transduction is induced by changing the medium of the cells to PBS containing 0.2% BSA, 10 mM LiCl, and the desired concentration of the oligopeptide, as determined by the screening. The cells are incubated in this medium for 5 minutes and then the media is removed from the cells. Next, 0.2 volumes ice-cold 20% (v/v) perchloric acid (PCA) is added to the cells to quench the stimulation and to prepare the cells for the inositol phosphate assay. The cells are incubated on ice in PCA for 20 minutes. At the beginning of the incubation, the cells are dislodged from the plate with a rubber policeman. After the incubation, the cells are removed from the plate and centrifuged at 2,000×g for 15 minutes at 4° C. The supernatants are removed and titrated to pH 7.5 with 10 N KOH and kept on ice. The solution is centrifuged at 2,000×g for 15 minutes at 4° C. to remove the precipitate. The supernatant is then assayed to determine the amount of inositol trisphosphate present.

Amersham provides a kit containing the reagents for an inositol triphosphate competition assay. With the kit, an inositol 1,4,5-trisphosphate binding protein is provided, which cross-reacts with inositol 1,3,4,5-tetrakisphosphate less than 10% and less than 1% with other inositol phosphates. The assay measures that amount of inositol triphosphate that competes for the binding protein with the radioactive labeled triphosphate.

1) Preparing the Standard

First allow all the reagents to thaw at 2-8° C. and then mix thoroughly. As this is occurring, eight poypropylene tubes (12×75 mm) are labeled “0.19,” “0.38,” “0.76,” “1.5,” “3.1,” 6.2, “12.5,” and “25 pmol.” 1.5 ml of water are pipetted into the tube marked “25 pmol.” Into the remaining marked standard tubes 500 μl of water are pipetted. Next, into the marked “25 pmol” tube, are added exactly 100 μl of the standard solution (3 nmol D-myo-inositol 1,4,5-trisphosphate in water). The solution is mixed completely. Five hundred μl of the 25 pmol solution are transferred to the 12.5 pmol tube, and the solution is vortexed throughly. Repeat this 1:2 dilution succesively with the remaining tubes. These working standards should be prepared immediately before each assay and not re-used. The standard solution from the kit should be recapped after use and immediately stored at −15° C. to −30° C.

2) Assay Protocol

First label duplicate polypropylene tubes (10×55 mm) “TC” for total counts; “NSB” for non-specific binding; “B₀” for zero standards; “0.19,” “0.38,” “0.76,” “1.5,” “3.1,” 6.2, “12.5,” and “25” pmol for the standards; and whatever is desired for the samples. Next, into all the tubes 100 μl of the assay buffer (0.1 M Tris buffer pH 9.0, 4 mM EDTA and 4 mg/ml bovine serum albumin (BSA)) is pipetted. Into the B₀ and TC tubes, 100 μl and 200 μl, respectively deionized water is added. Then, starting with the most dilute solution, 100 μl of each of the standard solutions, described above, is pipetted into the appropriately labelled tubes. A new pipette tip is used for each standard solution. Into the NSB tubes, 100 μl of stock standard solution (3 nmol D-myo-inositol 1,4,5-trisphosphate in water) are pipetted. One hundred microliters of the samples should be added to the appriopriate sample tubes. A new pipette tip is used for each sample.

One hundred microliters of first the tracer (˜1.0 μCi or ˜37 kBq of D-myo-[³H]inositol 1,4,5-trisphosphate in 1:1 (v/v) water:ethanol) and then binding protein is added to all the tubes. All the tubes are vortexed to mix all the contents throughly and then incubated for 15 minutes on ice. Then, the binding protein is isolated by the centrifugation procedure below.

All the tubes, except those labeled “TC”, are centrifuged at 2,000×g for at least 10 minutes at 4° C. After centrifugation, the tubes are carefully placed into a suitable decantation rack and the supernatant is poured off and discarded. The tubes are kept inverted and placed on absorbent tissues and allowed to drain for 2 minutes. Next, the rims of the inverted tubes are firmly blotted on the tissue to remove any adhering droplets of liquid, and the inside of the tubes are carefully swapped for the same reason. This is done carefully to avoid disturbing the pellet at the bottom of the tube.

To each tube, 200 μl of water is added to resuspend the pellet except the “TC” labeled tubes. The tube is vortexed to mix the solution throughly. Then, 2 ml of scintillation fluid is added to the resuspended pellet. Before measuring the radioactivity of each sample for four minutes in a γ-scintillation counter, the samples are capped and mixed throughly.

### Example 3

Purification of VSHK-1 Receptor Polypeptides from Nucleic Acids

The following protocol may be used for membrane preparation for ligand binding assay using COS-7, HEK293, or CHO cells transfected with an expression vector comprising VSHK-1-encoding sequences. COS-7 cells, for example, are grown in 245 mm×245 mm tissue culture plates and transfected with 30 μg of VSHK-1 receptor encoding plasmid DNA. After two days, the cells are washed with PBS and released from the culture plates by scraping the cells into PBS containing 5 mM EDTA and a protease cocktail. The protease cocktail contains 0.5 mM PMSF, 5 μg/ml aprotinin, 5 μg/ml leupeptin, and 5 μg/ml pepstatin. Cells are harvested by centrifugation 2,500×g for 5 minutes at 4° C., and resuspended in 1 ml of the PBS-protease cocktail. The harvested cells are lysed by rapidly diluting the cells into 20 ml of ice-cold 20 mM HEPES buffer, pH 7.5 containing protease cocktail. The lysed cells are centrifuged at 30,000 g for 30 minutes. The pellet, containing the cell membranes, is separated from the aqueous phase, and then the pellet is resuspended in 50 mM HEPES, pH 7.5, 10 mM MgCl₂. The membranes can be frozen at −70° C. for future use.

Deposit Information:

The following materials were deposited with the American Type Culture Collection:

| Name | Deposit Date | Accession No. |
|---|---|---|
| VCT170 in E. coli host DH5a | Oct. 30, 1998 | 98968 |
| VCT181 in E. coli host DH5a | Oct. 30, 1998 | 98967 |

VCT170 comprises full-length VSHK-1 coding sequence. VCT181 comprises 5′ untranslated region and partial coding sequence.

The above materials have been deposited with the American Type Culture Collection, Manassas, Va., U.S.A., under the accession numbers indicated. This deposit will be maintained under the terms of the Budapest Treaty on the International Recognition of the Deposit of Microorganisms for purposes of Patent Procedure. The deposits will be maintained for a period of 30 years following issuance of this patent, or for the enforceable life of the patent, whichever is greater. Upon issuance of the patent, the deposits will be available to the public from the ATCC without restriction.

These deposits are provided merely as convenience to those of skill in the art, and are not an admission that a deposit is required under 35 U.S.C. §112. The sequence of the polynucleotides contained within the deposited materials, as well as the amino acid sequence of the polypeptides encoded thereby, are incorporated herein by reference and are controlling in the event of any conflict with the written description of sequences herein. A license may be required to make, use, or sell the deposited materials, and no such license is granted hereby.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An isolated polypeptide comprising at least about 8 contiguous amino acids of the sequence set forth in amino acids 1-80 or amino acids 189-350 of SEQ ID NO:2.

2. The isolated polypeptide of claim 1, wherein said polypeptide has an amino acid sequence at least about 70% identical to the amino acid sequence depicted in SEQ ID NO:2

3. The isolated polypeptide of claim 1, wherein said polypeptide has the amino acid sequence depicted in SEQ ID NO:2.

4. A composition comprising the polypeptide of claim 1, and a buffer.

5 An isolated antibody specific for a VSHK-1 receptor polypeptide.

6. An isolated polynucleotide comprising nucleotide sequences which encode a polypeptide comprising at least about 8 contiguous amino acids of the sequence depicted in 1-80 or amino acids 189-350 SEQ ID NO:2

7. The isolated polynucleotide of claim 6, wherein said polynucleotide encodes a polypeptide comprising the sequence depicted in SEQ ID NO:2.

8. An isolated polynucleotide comprising a nucleotide sequence which has at least about 70% nucleotide sequence identity to nucleotides 1-324 or nucleotides 652-1890 of the sequence set forth in SEQ ID NO:1

9. The isolated polynucleotide of claim 8, wherein said polynucleotide hybridizes, under stringent conditions, with the polynucleotide sequence depicted in SEQ ID NO:1

10. An isolated polynucleotide comprising at least about 18 contiguous nucleotides of nucleotides 1-324 or 652-1890 of the sequence set forth in SEQ ID NO:1

11. The isolated polynucleotide of claim 8, wherein said polynucleotide comprises the nucleotide sequence depicted in SEQ ID NO:1

12. A recombinant vector comprising the polynucleotide of claim 8.

13. An isolated host cell comprising the polynucleotide of claim 12.

14. A method of detecting a VSHK-1 receptor polypeptide in a sample, comprising:
a) contacting the sample with an antibody which specifically binds the VSHK-1 receptor polypeptide; and
b) detecting binding between the antibody and molecules of the sample.

15. The method of claim 14, wherein said antibody is detectably labeled.

16. A method of detecting the presence of a VSHK-1 messenger RNA (mRNA) molecule in a biological sample, comprising:
a) contacting the sample with a VSHK-1 polynucleotide complementary to VSHK-1 mRNA under conditions which allow hybridization; and
b) detecting hybridization, if any.

17. A method of identifying a protein that binds to a VSHK-1 receptor polypeptide comprising:
(a) contacting said a test substance with a VSHK-1 receptor polypeptide under conditions that permit formation of a protein-protein complex; and
(b) detecting the presence of said complex.

18. A method of identifying a VSHK-1 receptor ligand, comprising:
(a) contacting a test substance with a VSHK-1 receptor polypeptide under conditions that permit formation of a ligand/receptor complex; and
(b) measuring a signal transduction activity of any complex formed.

19. A method of identifying a substance which modulates a signal transduction activity of a VSHK-1, comprising:
a) contacting the substance with a sample containing a VSHK-1 receptor polypeptide; and
b) assaying a signal transduction activity of the VSHK-1 receptor polypeptide in the presence of the substance, wherein an increase or a decrease in signal transduction activity in comparison to VSHK-1 signal transduction activity in a suitable control is an indication that the substance modulates a signal transduction activity of the VSHK-1.

20. The method of claim 18, wherein said signal transduction activity is measured in an intact cell by a measuring an intracellular level of a compound selected from the group consisting of inositol 1,4,5-triphosphate, diacylglycerol, and Ca²⁺.
